SEQUENCE LISTING (9 sequences)

SEQ ID NO: 1 - DNA, 2679 bp, Medicago truncatula
  source 1..2679: mol_type = genomic DNA; note = Mt12a genomic sequence
ccaaaacaacatttctttgtttagttgcaaaaataaccaatggagcctaactttatgttttcacttcttctcataatttctctttggcttctattaacctttcatagcaatgctgagacttccacatatataatccatatgaacaaatccttctttcctcaagtcttcacaaatcatcatgattggttcaaatctaccattcattccttaaaatcaaaaacattagtgcttgatgatcatgatcaacaagaagaagcatcaatgcaatcacagaagcaactagtatacacctatgataatgccatgtatggatttagtgcattgttatcttcaaatgagttagagactcttaacaacactgatggctttgttgcagcatatcaagacagaacagcaaccatggacacaactcacacctttgaatttctctccctagattctccaagtggtctatggcatgcttcaaatttcggagacgatatcattattggtgttattgactctggtgtatggcctgagagtcaaagctttaaagatgacggcatgacaaagaaaattccgaacaaatggaaaggaacatgtgaaactggccataaattcaatgcctccatgtgcaacttcaaactgattggagctagatctttcaacaaaggtgtgattgcttcaaatccaaatgttagaatcaggatgaactcagctagagacagtatcggacatggaactcatacatcatcaacagttgcaggtaactattttttattttttttatagagaaatgttagtactccttctgtaactctcagcttactaatcgagctgtctgcttcggacttcaggtaactatgtcaatggaacttcttattttggttatgctaaaggagtagcaagaggcattgcaccaagagctaggcttgctatgtacaaagtcatttgggaagagggtcttttggcttctgatgttttagctggaatggaccaagctattgctgatggtgttgatgtgatttcaatttctatgggatttgatggtgttccactttatgaagatgctatagcaatagcttcttttgcagctatggagaaagggattgttgtttcgtcttctgcaggtaactcaggacctaagcatggaactttgcacaatggtatcccttgggttcttactgtagctgcaggaaccatagatagaacttttggcagccttgttttggggaatggtcaaaacattattggttggactttgtttgcatcaaattcaactatagtcgaaaatcttcctcttgtttacgataacactttatcttcatgcaattcagtgaagcgtttatctcaagttaacaagcaagtcataattatttgtgactcaatatcaaactcaagctcagtgtttgatcaaatcgatgttgttacacaaaccaatatgttaggggcagtgtttctctctgatagtcctgaattaattgatttaagacatatctacgctccaggtattgttatcaagacaaaagatgcagaatctgtgatcaagtacgcaaaacgcaataagaataatccaacagcaagcattaagtttcaacaaacatttcttggaataaaaccggcaccaattgcagctcattactcatcaagaggtccatcacatggctttccttggattttgaagcctgacataatggcacctggatcaagagttctcgctgcttttgttccttataaaccaacagctagaattggtaccgatgtgttcttatcaagtgactacaatttcatgtcaggaacatccatggcttgtcctcatgcctctggtgttgctgctcttctcaaagctgtacatccacaatggagttctgctgctataagatctgcattgataaccacagctaatcctttggataatacaaaaaatctaataagggataacggttatccttctcaatatgcttcacctctcgctatcggtgctggtgagattgatcctaatagagcaatgaatccaggtttgatttatgatgctacaccacaagactatgttaatttcctttgtggtttgaagttcacaaagaatcaaattttaacgatcacaagatcaagttcttatggttgtgaaaacccttctttggatcttaattacccttcatttatagctttctacaacaaaaaaacaagatcaatggttcacacatttaacaggactgttaccaatgttggtgatggtgctgctacatatagtgctaatgtgacacatccaaaaggttgtgtgatgacagtgttacctgatatattaactttcaaatatagaaatgagaaacaaagttactcccttgtcataaagtgtgtcatgtacaagaaggataatgtttcatttggggatcttgtttggattgaatatggtggagcacacactgttagaagtcctattgtagtggcaccaaggggaattatatgagctttcaagtgttttttcccctcatttttctgcaccttctgcaatgacatgatatgcaatattgtgtacttttcttacactctttaaaaacactcattcaaatgcaaccttatgatgtgtttggttgtcattatcttggttttttattttcacaaactaattctacatcttcatgttttttgttctttaactactagttataacctcattggagaaccatcataagcttattt

SEQ ID NO: 2 - RNA, 2588 nt, Medicago truncatula
  source 1..2588: mol_type = mRNA; note = Mt12a mRNA
ccaaaacaacatttctttgtttagttgcaaaaataaccaatggagcctaactttatgttttcacttcttctcataatttctctttggcttctattaacctttcatagcaatgctgagacttccacatatataatccatatgaacaaatccttctttcctcaagtcttcacaaatcatcatgattggttcaaatctaccattcattccttaaaatcaaaaacattagtgcttgatgatcatgatcaacaagaagaagcatcaatgcaatcacagaagcaactagtatacacctatgataatgccatgtatggatttagtgcattgttatcttcaaatgagttagagactcttaacaacactgatggctttgttgcagcatatcaagacagaacagcaaccatggacacaactcacacctttgaatttctctccctagattctccaagtggtctatggcatgcttcaaatttcggagacgatatcattattggtgttattgactctggtgtatggcctgagagtcaaagctttaaagatgacggcatgacaaagaaaattccgaacaaatggaaaggaacatgtgaaactggccataaattcaatgcctccatgtgcaacttcaaactgattggagctagatctttcaacaaaggtgtgattgcttcaaatccaaatgttagaatcaggatgaactcagctagagacagtatcggacatggaactcatacatcatcaacagttgcaggtaactatgtcaatggaacttcttattttggttatgctaaaggagtagcaagaggcattgcaccaagagctaggcttgctatgtacaaagtcatttgggaagagggtcttttggcttctgatgttttagctggaatggaccaagctattgctgatggtgttgatgtgatttcaatttctatgggatttgatggtgttccactttatgaagatgctatagcaatagcttcttttgcagctatggagaaagggattgttgtttcgtcttctgcaggtaactcaggacctaagcatggaactttgcacaatggtatcccttgggttcttactgtagctgcaggaaccatagatagaacttttggcagccttgttttggggaatggtcaaaacattattggttggactttgtttgcatcaaattcaactatagtcgaaaatcttcctcttgtttacgataacactttatcttcatgcaattcagtgaagcgtttatctcaagttaacaagcaagtcataattatttgtgactcaatatcaaactcaagctcagtgtttgatcaaatcgatgttgttacacaaaccaatatgttaggggcagtgtttctctctgatagtcctgaattaattgatttaagacatatctacgctccaggtattgttatcaagacaaaagatgcagaatctgtgatcaagtacgcaaaacgcaataagaataatccaacagcaagcattaagtttcaacaaacatttcttggaataaaaccggcaccaattgcagctcattactcatcaagaggtccatcacatggctttccttggattttgaagcctgacataatggcacctggatcaagagttctcgctgcttttgttccttataaaccaacagctagaattggtaccgatgtgttcttatcaagtgactacaatttcatgtcaggaacatccatggcttgtcctcatgcctctggtgttgctgctcttctcaaagctgtacatccacaatggagttctgctgctataagatctgcattgataaccacagctaatcctttggataatacaaaaaatctaataagggataacggttatccttctcaatatgcttcacctctcgctatcggtgctggtgagattgatcctaatagagcaatgaatccaggtttgatttatgatgctacaccacaagactatgttaatttcctttgtggtttgaagttcacaaagaatcaaattttaacgatcacaagatcaagttcttatggttgtgaaaacccttctttggatcttaattacccttcatttatagctttctacaacaaaaaaacaagatcaatggttcacacatttaacaggactgttaccaatgttggtgatggtgctgctacatatagtgctaatgtgacacatccaaaaggttgtgtgatgacagtgttacctgatatattaactttcaaatatagaaatgagaaacaaagttactcccttgtcataaagtgtgtcatgtacaagaaggataatgtttcatttggggatcttgtttggattgaatatggtggagcacacactgttagaagtcctattgtagtggcaccaaggggaattatatgagctttcaagtgttttttcccctcatttttctgcaccttctgcaatgacatgatatgcaatattgtgtacttttcttacactctttaaaaacactcattcaaatgcaaccttatgatgtgtttggttgtcattatcttggttttttattttcacaaactaattctacatcttcatgttttttgttctttaactactagttataacctcattggagaaccatcataagcttattt

SEQ ID NO: 3 - DNA, 2316 bp, Medicago truncatula
  source 1..2316: mol_type = other DNA; note = Encodes for the amino acid sequence in SEQ ID NO:5; note = Mt12a coding sequence
atggagcctaactttatgttttcacttcttctcataatttctctttggcttctattaacctttcatagcaatgctgagacttccacatatataatccatatgaacaaatccttctttcctcaagtcttcacaaatcatcatgattggttcaaatctaccattcattccttaaaatcaaaaacattagtgcttgatgatcatgatcaacaagaagaagcatcaatgcaatcacagaagcaactagtatacacctatgataatgccatgtatggatttagtgcattgttatcttcaaatgagttagagactcttaacaacactgatggctttgttgcagcatatcaagacagaacagcaaccatggacacaactcacacctttgaatttctctccctagattctccaagtggtctatggcatgcttcaaatttcggagacgatatcattattggtgttattgactctggtgtatggcctgagagtcaaagctttaaagatgacggcatgacaaagaaaattccgaacaaatggaaaggaacatgtgaaactggccataaattcaatgcctccatgtgcaacttcaaactgattggagctagatctttcaacaaaggtgtgattgcttcaaatccaaatgttagaatcaggatgaactcagctagagacagtatcggacatggaactcatacatcatcaacagttgcaggtaactatgtcaatggaacttcttattttggttatgctaaaggagtagcaagaggcattgcaccaagagctaggcttgctatgtacaaagtcatttgggaagagggtcttttggcttctgatgttttagctggaatggaccaagctattgctgatggtgttgatgtgatttcaatttctatgggatttgatggtgttccactttatgaagatgctatagcaatagcttcttttgcagctatggagaaagggattgttgtttcgtcttctgcaggtaactcaggacctaagcatggaactttgcacaatggtatcccttgggttcttactgtagctgcaggaaccatagatagaacttttggcagccttgttttggggaatggtcaaaacattattggttggactttgtttgcatcaaattcaactatagtcgaaaatcttcctcttgtttacgataacactttatcttcatgcaattcagtgaagcgtttatctcaagttaacaagcaagtcataattatttgtgactcaatatcaaactcaagctcagtgtttgatcaaatcgatgttgttacacaaaccaatatgttaggggcagtgtttctctctgatagtcctgaattaattgatttaagacatatctacgctccaggtattgttatcaagacaaaagatgcagaatctgtgatcaagtacgcaaaacgcaataagaataatccaacagcaagcattaagtttcaacaaacatttcttggaataaaaccggcaccaattgcagctcattactcatcaagaggtccatcacatggctttccttggattttgaagcctgacataatggcacctggatcaagagttctcgctgcttttgttccttataaaccaacagctagaattggtaccgatgtgttcttatcaagtgactacaatttcatgtcaggaacatccatggcttgtcctcatgcctctggtgttgctgctcttctcaaagctgtacatccacaatggagttctgctgctataagatctgcattgataaccacagctaatcctttggataatacaaaaaatctaataagggataacggttatccttctcaatatgcttcacctctcgctatcggtgctggtgagattgatcctaatagagcaatgaatccaggtttgatttatgatgctacaccacaagactatgttaatttcctttgtggtttgaagttcacaaagaatcaaattttaacgatcacaagatcaagttcttatggttgtgaaaacccttctttggatcttaattacccttcatttatagctttctacaacaaaaaaacaagatcaatggttcacacatttaacaggactgttaccaatgttggtgatggtgctgctacatatagtgctaatgtgacacatccaaaaggttgtgtgatgacagtgttacctgatatattaactttcaaatatagaaatgagaaacaaagttactcccttgtcataaagtgtgtcatgtacaagaaggataatgtttcatttggggatcttgtttggattgaatatggtggagcacacactgttagaagtcctattgtagtggcaccaaggggaattatatga

SEQ ID NO: 4 - DNA, 2000 bp, Medicago truncatula
  source 1..2000: mol_type = genomic DNA; note = Mt12a promoter (2kb)
caaacggggtcataagagtttttttttttaataaagactaagagataatttccatcaaagaagcagattgcaagaatgtcggataaaaaaaggaagaataaaaaaaaaattgaacaagcaaaaaggaaaaataaaatgatggccatgacactgcaaaagaaaatgaagcaaacacaatttaagcatctcttaactcaggaatttcttcttcctaacataaaaactaactaatcagatttattaactttcttagatccatttttgcttataatttaagatagagggagtttgatttttttttttttttgttataagagggagtttgaatttaggattaagaaatatgttgaattaacatattaaaataatgtcatccttaggcacagaaaatattagttctttgtagtttaaatctctttgggctacacaaagaatgttaaaatatcgccatccttaggtacagaaaaataataacatccttattatggctattctatcaaccaaaaactcattattatgtgatgccaattgactcctcaagagtccagagatttgtccatctctaacataagttacataattaattatttgatgttccggtgggttcgacaccttttgacttgtacaagcaattcaacaaaaaaacaataaggtgagatacagttttctttttttattatttggttgtcgattgatgcacaccaaaattaactttttttttttttttttaaggaaaattaactttaaaaatatggagtcaaatacattaatgtaatgtagtttatcccttaactttatttgatatgatcaactagatatctctttgcacaattgtagatgttaatcccctcagcttcgtaggactgtcatttttgatatagttttccttcaactaacttcataaatagagactccattcttttacttatttattttgtagttatttaagatgatacatcggtcgttaacttttctatacaatggtggtctttgacaccccttcctttaggacctttcttttatagggatagatgtcatttttttgtatagattatcctctctatgactctgttgtattggttcagacatcttctgtcattttatttttattttagtgttttgataaaataaaataaaaatgatacaatttcttatattcaacaatatcacaattgaaggaccttccaaaaaatatcacaataataatatagttagtattaaaatgattaaaatgctatgctacatttttcctcacacaaagttttcttcattttcagcattaatatcatttataaattttcattataccaactgcaaatttcaaataccacctaaaattaagaattttaagttttcattaatctaattttaaatttttgttaaactagtatactacgctttaattattatagtccaacccatctaggtttgtatatatgcacggtgatattccatgggatttgaagtatgcttctctcaagatttgaggtttaattttccgatgtcaatttcggtgaactagtacacacataccctttttcttattttaaatgggtgcctgtaagtgaatagtggaattgaacctttggaattagtcggtctttgtgtcgaatatcaagtttttaaaaaaaaataataataaaattggacacacacattaataattaatttagataaaagtttatacatttctacccttaattcatttattacatgattttggtttttgaaaggatacaaaagaaaaacatcattgtatcttggtttcctaaaacataaccaaagtcttaggacaaaaataaaaagcttaaaggaccaaagattagagagggacgaagtactgttaagcaagaaagtttcttcaatcatttagaagtaatatcatcaaataatttatgaatgtctacatcattgatattctctaaaaatcctatataatcacatgtaagaggaatcaatagccaaaacaacatttctttgtttagttgcaaaaataacca

SEQ ID NO: 5 - protein, 771 aa, Medicago truncatula
  source 1..771: note = Is the amino acid sequence encoded by SEQ ID NO:3
MEPNFMFSLLLIISLWLLLTFHSNAETSTYIIHMNKSFFPQVFTNHHDWFKSTIHSLKSKTLVLDDHDQQEEASMQSQKQLVYTYDNAMYGFSALLSSNELETLNNTDGFVAAYQDRTATMDTTHTFEFLSLDSPSGLWHASNFGDDIIIGVIDSGVWPESQSFKDDGMTKKIPNKWKGTCETGHKFNASMCNFKLIGARSFNKGVIASNPNVRIRMNSARDSIGHGTHTSSTVAGNYVNGTSYFGYAKGVARGIAPRARLAMYKVIWEEGLLASDVLAGMDQAIADGVDVISISMGFDGVPLYEDAIAIASFAAMEKGIVVSSSAGNSGPKHGTLHNGIPWVLTVAAGTIDRTFGSLVLGNGQNIIGWTLFASNSTIVENLPLVYDNTLSSCNSVKRLSQVNKQVIIICDSISNSSSVFDQIDVVTQTNMLGAVFLSDSPELIDLRHIYAPGIVIKTKDAESVIKYAKRNKNNPTASIKFQQTFLGIKPAPIAAHYSSRGPSHGFPWILKPDIMAPGSRVLAAFVPYKPTARIGTDVFLSSDYNFMSGTSMACPHASGVAALLKAVHPQWSSAAIRSALITTANPLDNTKNLIRDNGYPSQYASPLAIGAGEIDPNRAMNPGLIYDATPQDYVNFLCGLKFTKNQILTITRSSSYGCENPSLDLNYPSFIAFYNKKTRSMVHTFNRTVTNVGDGAATYSANVTHPKGCVMTVLPDILTFKYRNEKQSYSLVIKCVMYKKDNVSFGDLVWIEYGGAHTVRSPIVVAPRGII

SEQ ID NO: 6 - DNA, 7741 bp, Medicago truncatula
  source 1..7741: mol_type = genomic DNA; note = Is the Sbt12a nucleotide sequence in the Sbt12a-1 mutant.
atggagcctaactttatgttttcacttcttctcataatttctctttggcttctattaacctttcatagcaatgctgagacttccacatatataatccatatgaacaaatccttctttcctcaagtcttcacaaatcatcatgattggttcaaatctaccattcattccttaaaatcaaaaacattagtgcttgatgatcatgatcaacaagaagaagcatcaatgcaatcacagaagcaactagtatatgatgatgtccatctcattgaagaagtattaggcatgtgcctaataagagttttctttggtttggtagccaaccttgttgacttggtttggttggtagccaaccttgttgaatccttgttggattggtagccaactttgttgaattgtgaaaaatgtgtgtaaattgtcaaatattgtaggctttagagggtgaagctttggctataaaaggagagcttcaactctcatttcttcacaccaacaaagagagaaagaaagagtgaggtttcacagacaaggtataagaaaatagtctgtgaggaaaatagagagtgagcgatattgtagtgaggtgggaatatcaaaagagggttatttcttttgagtgttgtagtggtctttggagtatttacctccgacctacaaagtgtaaaattccttactatagtgatatcagttgctcctctcggggtcgtggttttttttcccttattcagaagggttttccacgtaaaaatcttggtgtcattgttactcttttattcttgttaattaccgtatctcggtgctacattattattccgctttattaccgtgaatattattttggtaaggggtttattcccaacaactggtatcagagcacaggttctgctcgttcactgaaatactattcactgtcggtagtactatacttggtgaaaaataaaaatgtctggagtaaagtacgaggtagcaaaattcaatggagataacggtttctcaacatggcaaagaaggatgagagatctgctcatccaacaaggattacacaaggttctagatgttgattccaaaaagcctgataccatgaaagctgaggattgggctgacttggatgaaagagctgctagtgcaatcaggttgcacttatcagatgatgtggtaaataacatcattgatgaagacactgcacgtggaatttggacaaggttggaaagcctatacatgtccaaaacgctgacaaataaattgtacctgaagaagcagttatacgccctacacatgagtgaaggtacgaattttttgtcacatttaaatgtgtttaacggactaatcacacagcttgccaacctcggagtgaaaatcgaggaagaagataaagccatcttgctattgaactcgttgccatcttcgtacgataatttggcaacaaccatcctgcacggtaagactactattgagttgaaagatgtcacatcggctcttctactcaatgagaagatgagaaagaagcctgaaaatcaaggacaggctctcatcacagaaggtagaggcaggagttatcaaaggagttcgaacaactatggtagatccggagctcgtgggaagtcaaagaatcgatccaaatcaagagtcagaaattgctacaactgtaatcaaccaggtcacttcaaaagagattgcccaaatccaaggaagggcaaaggtgaaaccagtggccagaagaatgacgacaacacagccgccatggtgcaaaataatgataatgttgtcctctttataaatgaggaagaggaatgcatgcacctgtcaggtccagagtcggaatgggtggttgacacagcggcatctcaccatgccacaccggtaagagatcttttttgcagatatgtagcaggtgatttcggcacagtgaaaatgggtaacacaagttactcaaagattgcggggattggtgacatttgtatcaagacaaatgtcggatgcacattggttctaaaggatgtgcggcatgtacctgatttgcggatgaacttgatctcgggaattgctttagaccgagatggatacgagagttattttgcaaatcaaaagtggagactcactaagggatcattggtgattgcaaagggagttgctcgtggcacgttgtacaggacaaatgcagaaatatgccaaggtgaattgaacgcggcacaagatgagatttctgtagatttatggcacaaaagaatgggtcatatgagcgagaagggattgcagattcttgccaagaaatcactcatttcttatgccaaaggtacaactgtaaaaccttgtgactactgtttatttggtaagcagcatagagtctcatttcagacatcgtctgaaagaaaattgaatatacttgatttagtatattctgatgtttgcggcccaatggaaattgaatcaatgggcggtaacaaatattttgttacttttattgatgatgcttcacgaaaattatgggtttatattttgaaaaccaaagatcaggtgtttcaagttttccagaagtttcatgctctagtagaaagggagacgggtcgaaagctaaagcgtctccgaagtgacaatggaggtgagtacacttcaagggaatttgaagagtattgttcaagtcatgggatcagacatgaaaagacagttcctggaaccccacagcacaatggcgtagccgagaggatgaaccgcaccattgtggagaaggtgagaagcatgctcagaatggctaaactgcctaagtcattctggggtgaagcagttcagacagcctgttacctgatcaataggagtccatcagttccgttggcgtttgaaatcccagagagagtctggaccaacaaggaggtgtcctactcgcatctgaaggtgttcggttgcagagcttttgcacatgtaccaaaagagcagagaacaaagctggatgataaatctattccctgcatatttatcggatatggagatgaagagttcgggtacagactgtgggatcctgtaaagaagaaggtcatcagaagtagagatgtagtcttccgagaaagtgaagttagaactgctgctgatatgtcagaaaaggtgaagaatggtataattcctaactttgttactattccttctacttctaacaatcccacaagtgcagaaagtacgaccgacgaggtttccgagcagggggagcaacctggtgaggttattgagcagggggagcaacttgatgaaggtgtcgaggaagtggagcaccccactcagggagaagaacaacatcaacctctgaggagatcagagaggccaagggtagagtcacgcaggtacccttccacagagtatgtcctcatcagtgatgatagggagccagaaagtcttaaggaggtgttgtcccatccagaaaagaaccagttgatgaaagctatgcaagaagagatggaatctctccagaaaaatggcacatacaagctggttgaacttccaaagggtaaaagaccactcaaatgcaaatgggtctttaaactcaagaaagatggagattgcaagctggtcagatacaaagctcgattggtggttaaaggcttcgaacagaagaaaggtattgattttgacgaaattttctcccccgttgttaaaatgacttctattcgaacaattttgagcttagcagctagcctagatcttgaagtggagcagttggatgtgaaaactgcatttcttcatggagatttggaagaggagatttatatggagcaaccagaaggatttgaagtagctggaaagaaacacatggtgtgcaaattgaataagagtctttatggattgaagcaggcaccaaggcagtggtacatgaagtttgattcattcatgaaaagtcaaacatacctaaagacctattctgatccatgtgtatacttcaaaagattttctgagaataactttattatattgttgttgtatgtggatgacatgctaattgtaggaaaagacaaggggttgatagcaaagttgaaaggagatctgtccaagtcatttgatatgaaggacttgggcccagcacaacaaattctagggatgaagatagttcgagagagaacaagtagaaagttgtggctatctcaggagaagtacattgaacgtgtactagaacgcttcaacatgaagaatgctaagccagtcagcacacctcttgctggtcatctaaagttgagtaaaaagatgtgtcctacaacagtggaagagaaagggaacatggctaaagttccttattcttcagcagtcggaagcttgatgtatgcaatggtatgtactagacctgatattgctcacgcagttggtgttgtcagcaggtttcttgaaaatcctggaaaggaacattgggaagcagtcaagtggatactcaggtacctgagaggtaccacgggagattgtttgtgctttggaggatctgatccaatcttgaagggctatacagatgctgatatggcaggtgacattgacaacagaaaatccagtactggatatttgtttacattttcagggggagctatatcatggcagtctaagttgcaaaagtgcgttgcactttcaacaactgaagcagagtacattgctgctacagaaactggcaaggagatgatatggctcaagcgattccttcaagagcttggattgcatcagaaggagtatgtcgtctattgtgacagtcaaagtgcaatagaccttagcaagaactctatgtaccatgcaaggaccaaacacattgatgtgagatatcattggattcgagaaatggtagatgatgaatctctaaaagtcttgaagatttctacaaatgagaatcccgcagatatgctgaccaaggtggtaccaaggaacaagttcgagctatgcaaagaacttgtcggcatgcattcaaactagaagacagtgctacctcctctggatgaatgagactggagggggagattgatgatgtccatctcattgaagaagtattaggcatgtgcctaataagagttttctttggtttggtagccaaccttgttgacttggtttggttggtagccaaccttgttgaatccttgttggattggtagccaactttgttgaattgtgaaaaatgtgtgtaaattgtcaaatattgtaggctttagagggtgaagctttggctataaaaggagagcttcaactctcatttcttcacaccaacaaagagagaaagaaagagtgaggtttcacagacaaggtataagaaaatagtctgtgaggaaaatagagagtgagcgatattgtagtgaggtgggaatatcaaaagagggttatttcttttgagtgttgtagtggtctttggagtatttacctccgacctacaaagtgtaaaattccttactatagtgatatcagttgctcctctcggggtcgtggttttttttcccttattcagaagggttttccacgtaaaaatcttggtgtcattgttactcttttattcttgttaattaccgtatctcggtgctacattattattccgctttattaccgtgaatattattttggtaaggggtttattcccaacacacctatgataatgccatgtatggatttagtgcattgttatcttcaaatgagttagagactcttaacaacactgatggctttgttgcagcatatcaagacagaacagcaaccatggacacaactcacacctttgaatttctctccctagattctccaagtggtctatggcatgcttcaaatttcggagacgatatcattattggtgttattgactctggtgtatggcctgagagtcaaagctttaaagatgacggcatgacaaagaaaattccgaacaaatggaaaggaacatgtgaaactggccataaattcaatgcctccatgtgcaacttcaaactgattggagctagatctttcaacaaaggtgtgattgcttcaaatccaaatgttagaatcaggatgaactcagctagagacagtatcggacatggaactcatacatcatcaacagttgcaggtaactattttttattttttttatagagaaatgttagtactccttctgtaactctcagcttactaatcgagctgtctgcttcggacttcaggtaactatgtcaatggaacttcttattttggttatgctaaaggagtagcaagaggcattgcaccaagagctaggcttgctatgtacaaagtcatttgggaagagggtcttttggcttctgatgttttagctggaatggaccaagctattgctgatggtgttgatgtgatttcaatttctatgggatttgatggtgttccactttatgaagatgctatagcaatagcttcttttgcagctatggagaaagggattgttgtttcgtcttctgcaggtaactcaggacctaagcatggaactttgcacaatggtatcccttgggttcttactgtagctgcaggaaccatagatagaacttttggcagccttgttttggggaatggtcaaaacattattggttggactttgtttgcatcaaattcaactatagtcgaaaatcttcctcttgtttacgataacactttatcttcatgcaattcagtgaagcgtttatctcaagttaacaagcaagtcataattatttgtgactcaatatcaaactcaagctcagtgtttgatcaaatcgatgttgttacacaaaccaatatgttaggggcagtgtttctctctgatagtcctgaattaattgatttaagacatatctacgctccaggtattgttatcaagacaaaagatgcagaatctgtgatcaagtacgcaaaacgcaataagaataatccaacagcaagcattaagtttcaacaaacatttcttggaataaaaccggcaccaattgcagctcattactcatcaagaggtccatcacatggctttccttggattttgaagcctgacataatggcacctggatcaagagttctcgctgcttttgttccttataaaccaacagctagaattggtaccgatgtgttcttatcaagtgactacaatttcatgtcaggaacatccatggcttgtcctcatgcctctggtgttgctgctcttctcaaagctgtacatccacaatggagttctgctgctataagatctgcattgataaccacagctaatcctttggataatacaaaaaatctaataagggataacggttatccttctcaatatgcttcacctctcgctatcggtgctggtgagattgatcctaatagagcaatgaatccaggtttgatttatgatgctacaccacaagactatgttaatttcctttgtggtttgaagttcacaaagaatcaaattttaacgatcacaagatcaagttcttatggttgtgaaaacccttctttggatcttaattacccttcatttatagctttctacaacaaaaaaacaagatcaatggttcacacatttaacaggactgttaccaatgttggtgatggtgctgctacatatagtgctaatgtgacacatccaaaaggttgtgtgatgacagtgttacctgatatattaactttcaaatatagaaatgagaaacaaagttactcccttgtcataaagtgtgtcatgtacaagaaggataatgtttcatttggggatcttgtttggattgaatatggtggagcacacactgttagaagtcctattgtagtggcaccaaggggaattatatga

SEQ ID NO: 7 - protein, 125 aa, Medicago truncatula
  source 1..125: note = Is the Sbt12a amino acid sequence in the Sbt12a-1 mutant.
MEPNFMFSLLLIISLWLLLTFHSNAETSTYIIHMNKSFFPQVFTNHHDWFKSTIHSLKSKTLVLDDHDQQEEASMQSQKQLVYDDVHLIEEVLGMCLIRVFFGLVANLVDLVWLVANLVESLLDW

SEQ ID NO: 8 - DNA, 7741 bp, Medicago truncatula
  source 1..7741: mol_type = genomic DNA; note = Is the Sbt12a nucleotide sequence in the Sbt12a-2 mutant.
atggagcctaactttatgttttcacttcttctcataatttctctttggcttctattaacctttcatagcaatgctgagacttccacatatataatccatatgaacaaatccttctttcctcaagtcttcacaaatcatcatgattggttcaaatctaccattcattccttaaaatcaaaaacattagtgcttgatgatcatgatcaacaagaagaagcatcaatgcaatcacagaagcaactagtatacacctatgataatgccatgtatggatttagtgcattgttatcttcaaatgagttagagactcttaacaacactgatggctttgttgcagcatatcaagacagaacagcaaccatggacacaactcacacctttgaatttctctccctagattctccaagtggtctatggcatgcttcaaatttcggagacgatatcattattggtgttattgactctggtgtatggcctgagagtcaaagctttaaagatgacggcatgacaaagaaaattccgaacaaatggaaaggaacatgtgaaactggccataaattcaatgcctccatgtgcaacttcaaactgattggagctagatctttcaacaaaggtgtgattgcttcaaatccaaatgttagaatcaggatgaactcagctagagacagtatcggacatggaactcatacatcatcaacagttgcaggtaactattttttattttttttatagagaaatgttagtactccttctgtaactctcagcttactaatcgagctgtctgcttcggacttcaggtaactatgtcaatggaacttcttattttggttatgctaaaggagtagcaagaggcattgcaccaagagctaggcttgctatgtacaaagtcatttgggaagagggtcttttggcttctgatgttttagctggaatggaccaagctattgctgatggtgttgatgtgatttcaatttctatgggatttgatggtgttccactttatgaagatgctatagcaatagcttcttttgcagctatggagaaagggattgttgtttcgtcttctgcaggtaactcaggacctaagcatggaactttgcacaatggtatcccttgggttcttactgtagctgcaggaaccatagatagaacttttggcagccttgttttggggaatggtcaaaacattattgtgatgatgtccatctcattgaagaagtattaggcatgtgcctaataagagttttctttggtttggtagccaaccttgttgacttggtttggttggtagccaaccttgttgaatccttgttggattggtagccaactttgttgaattgtgaaaaatgtgtgtaaattgtcaaatattgtaggctttagagggtgaagctttggctataaaaggagagcttcaactctcatttcttcacaccaacaaagagagaaagaaagagtgaggtttcacagacaaggtataagaaaatagtctgtgaggaaaatagagagtgagcgatattgtagtgaggtgggaatatcaaaagagggttatttcttttgagtgttgtagtggtctttggagtatttacctccgacctacaaagtgtaaaattccttactatagtgatatcagttgctcctctcggggtcgtggttttttttcccttattcagaagggttttccacgtaaaaatcttggtgtcattgttactcttttattcttgttaattaccgtatctcggtgctacattattattccgctttattaccgtgaatattattttggtaaggggtttattcccaacaactggtatcagagcacaggttctgctcgttcactgaaatactattcactgtcggtagtactatacttggtgaaaaataaaaatgtctggagtaaagtacgaggtagcaaaattcaatggagataacggtttctcaacatggcaaagaaggatgagagatctgctcatccaacaaggattacacaaggttctagatgttgattccaaaaagcctgataccatgaaagctgaggattgggctgacttggatgaaagagctgctagtgcaatcaggttgcacttatcagatgatgtggtaaataacatcattgatgaagacactgcacgtggaatttggacaaggttggaaagcctatacatgtccaaaacgctgacaaataaattgtacctgaagaagcagttatacgccctacacatgagtgaaggtacgaattttttgtcacatttaaatgtgtttaacggactaatcacacagcttgccaacctcggagtgaaaatcgaggaagaagataaagccatcttgctattgaactcgttgccatcttcgtacgataatttggcaacaaccatcctgcacggtaagactactattgagttgaaagatgtcacatcggctcttctactcaatgagaagatgagaaagaagcctgaaaatcaaggacaggctctcatcacagaaggtagaggcaggagttatcaaaggagttcgaacaactatggtagatccggagctcgtgggaagtcaaagaatcgatccaaatcaagagtcagaaattgctacaactgtaatcaaccaggtcacttcaaaagagattgcccaaatccaaggaagggcaaaggtgaaaccagtggccagaagaatgacgacaacacagccgccatggtgcaaaataatgataatgttgtcctctttataaatgaggaagaggaatgcatgcacctgtcaggtccagagtcggaatgggtggttgacacagcggcatctcaccatgccacaccggtaagagatcttttttgcagatatgtagcaggtgatttcggcacagtgaaaatgggtaacacaagttactcaaagattgcggggattggtgacatttgtatcaagacaaatgtcggatgcacattggttctaaaggatgtgcggcatgtacctgatttgcggatgaacttgatctcgggaattgctttagaccgagatggatacgagagttattttgcaaatcaaaagtggagactcactaagggatcattggtgattgcaaagggagttgctcgtggcacgttgtacaggacaaatgcagaaatatgccaaggtgaattgaacgcggcacaagatgagatttctgtagatttatggcacaaaagaatgggtcatatgagcgagaagggattgcagattcttgccaagaaatcactcatttcttatgccaaaggtacaactgtaaaaccttgtgactactgtttatttggtaagcagcatagagtctcatttcagacatcgtctgaaagaaaattgaatatacttgatttagtatattctgatgtttgcggcccaatggaaattgaatcaatgggcggtaacaaatattttgttacttttattgatgatgcttcacgaaaattatgggtttatattttgaaaaccaaagatcaggtgtttcaagttttccagaagtttcatgctctagtagaaagggagacgggtcgaaagctaaagcgtctccgaagtgacaatggaggtgagtacacttcaagggaatttgaagagtattgttcaagtcatgggatcagacatgaaaagacagttcctggaaccccacagcacaatggcgtagccgagaggatgaaccgcaccattgtggagaaggtgagaagcatgctcagaatggctaaactgcctaagtcattctggggtgaagcagttcagacagcctgttacctgatcaataggagtccatcagttccgttggcgtttgaaatcccagagagagtctggaccaacaaggaggtgtcctactcgcatctgaaggtgttcggttgcagagcttttgcacatgtaccaaaagagcagagaacaaagctggatgataaatctattccctgcatatttatcggatatggagatgaagagttcgggtacagactgtgggatcctgtaaagaagaaggtcatcagaagtagagatgtagtcttccgagaaagtgaagttagaactgctgctgatatgtcagaaaaggtgaagaatggtataattcctaactttgttactattccttctacttctaacaatcccacaagtgcagaaagtacgaccgacgaggtttccgagcagggggagcaacctggtgaggttattgagcagggggagcaacttgatgaaggtgtcgaggaagtggagcaccccactcagggagaagaacaacatcaacctctgaggagatcagagaggccaagggtagagtcacgcaggtacccttccacagagtatgtcctcatcagtgatgatagggagccagaaagtcttaaggaggtgttgtcccatccagaaaagaaccagttgatgaaagctatgcaagaagagatggaatctctccagaaaaatggcacatacaagctggttgaacttccaaagggtaaaagaccactcaaatgcaaatgggtctttaaactcaagaaagatggagattgcaagctggtcagatacaaagctcgattggtggttaaaggcttcgaacagaagaaaggtattgattttgacgaaattttctcccccgttgttaaaatgacttctattcgaacaattttgagcttagcagctagcctagatcttgaagtggagcagttggatgtgaaaactgcatttcttcatggagatttggaagaggagatttatatggagcaaccagaaggatttgaagtagctggaaagaaacacatggtgtgcaaattgaataagagtctttatggattgaagcaggcaccaaggcagtggtacatgaagtttgattcattcatgaaaagtcaaacatacctaaagacctattctgatccatgtgtatacttcaaaagattttctgagaataactttattatattgttgttgtatgtggatgacatgctaattgtaggaaaagacaaggggttgatagcaaagttgaaaggagatctgtccaagtcatttgatatgaaggacttgggcccagcacaacaaattctagggatgaagatagttcgagagagaacaagtagaaagttgtggctatctcaggagaagtacattgaacgtgtactagaacgcttcaacatgaagaatgctaagccagtcagcacacctcttgctggtcatctaaagttgagtaaaaagatgtgtcctacaacagtggaagagaaagggaacatggctaaagttccttattcttcagcagtcggaagcttgatgtatgcaatggtatgtactagacctgatattgctcacgcagttggtgttgtcagcaggtttcttgaaaatcctggaaaggaacattgggaagcagtcaagtggatactcaggtacctgagaggtaccacgggagattgtttgtgctttggaggatctgatccaatcttgaagggctatacagatgctgatatggcaggtgacattgacaacagaaaatccagtactggatatttgtttacattttcagggggagctatatcatggcagtctaagttgcaaaagtgcgttgcactttcaacaactgaagcagagtacattgctgctacagaaactggcaaggagatgatatggctcaagcgattccttcaagagcttggattgcatcagaaggagtatgtcgtctattgtgacagtcaaagtgcaatagaccttagcaagaactctatgtaccatgcaaggaccaaacacattgatgtgagatatcattggattcgagaaatggtagatgatgaatctctaaaagtcttgaagatttctacaaatgagaatcccgcagatatgctgaccaaggtggtaccaaggaacaagttcgagctatgcaaagaacttgtcggcatgcattcaaactagaagacagtgctacctcctctggatgaatgagactggagggggagattgatgatgtccatctcattgaagaagtattaggcatgtgcctaataagagttttctttggtttggtagccaaccttgttgacttggtttggttggtagccaaccttgttgaatccttgttggattggtagccaactttgttgaattgtgaaaaatgtgtgtaaattgtcaaatattgtaggctttagagggtgaagctttggctataaaaggagagcttcaactctcatttcttcacaccaacaaagagagaaagaaagagtgaggtttcacagacaaggtataagaaaatagtctgtgaggaaaatagagagtgagcgatattgtagtgaggtgggaatatcaaaagagggttatttcttttgagtgttgtagtggtctttggagtatttacctccgacctacaaagtgtaaaattccttactatagtgatatcagttgctcctctcggggtcgtggttttttttcccttattcagaagggttttccacgtaaaaatcttggtgtcattgttactcttttattcttgttaattaccgtatctcggtgctacattattattccgctttattaccgtgaatattattttggtaaggggtttattcccaacagttggactttgtttgcatcaaattcaactatagtcgaaaatcttcctcttgtttacgataacactttatcttcatgcaattcagtgaagcgtttatctcaagttaacaagcaagtcataattatttgtgactcaatatcaaactcaagctcagtgtttgatcaaatcgatgttgttacacaaaccaatatgttaggggcagtgtttctctctgatagtcctgaattaattgatttaagacatatctacgctccaggtattgttatcaagacaaaagatgcagaatctgtgatcaagtacgcaaaacgcaataagaataatccaacagcaagcattaagtttcaacaaacatttcttggaataaaaccggcaccaattgcagctcattactcatcaagaggtccatcacatggctttccttggattttgaagcctgacataatggcacctggatcaagagttctcgctgcttttgttccttataaaccaacagctagaattggtaccgatgtgttcttatcaagtgactacaatttcatgtcaggaacatccatggcttgtcctcatgcctctggtgttgctgctcttctcaaagctgtacatccacaatggagttctgctgctataagatctgcattgataaccacagctaatcctttggataatacaaaaaatctaataagggataacggttatccttctcaatatgcttcacctctcgctatcggtgctggtgagattgatcctaatagagcaatgaatccaggtttgatttatgatgctacaccacaagactatgttaatttcctttgtggtttgaagttcacaaagaatcaaattttaacgatcacaagatcaagttcttatggttgtgaaaacccttctttggatcttaattacccttcatttatagctttctacaacaaaaaaacaagatcaatggttcacacatttaacaggactgttaccaatgttggtgatggtgctgctacatatagtgctaatgtgacacatccaaaaggttgtgtgatgacagtgttacctgatatattaactttcaaatatagaaatgagaaacaaagttactcccttgtcataaagtgtgtcatgtacaagaaggataatgtttcatttggggatcttgtttggattgaatatggtggagcacacactgttagaagtcctattgtagtggcaccaaggggaattatatga

SEQ ID NO: 9 - protein, 243 aa, Medicago truncatula
  source 1..243: note = Is the Sbt12a amino acid sequence in the Sbt12a-2 mutant.
MEPNFMFSLLLIISLWLLLTFHSNAETSTYIIHMNKSFFPQVFTNHHDWFKSTIHSLKSKTLVLDDHDQQEEASMQSQKQLVYTYDNAMYGFSALLSSNELETLNNTDGFVAAYQDRTATMDTTHTFEFLSLDSPSGLWHASNFGDDIIIGVIDSGVWPESQSFKDDGMTKKIPNKWKGTCETGHKFNASMCNFKLIGARSFNKGVIASNPNVRIRMNSARDSIGHGTHTSSTVAGNYFLFFL

(19) United States
(12) Patent Application Publication
(10) Pub. No.: US 2025/0075226 A1
(43) Pub. Date: Mar. 6, 2025

(54) PROTEINS FOR REGULATION OF SYMBIOTIC INFECTION AND ASSOCIATED REGULATORY ELEMENTS

(71) Applicant: University of Freiburg, Freiburg (DE)

(72) Inventors: Thomas Ott, Merzhausen (DE); Guofeng Zhang, Freiburg (DE)

(21) Appl. No.: 18/818,545

(22) Filed: Aug. 28, 2024

Related U.S. Application Data
(60) Provisional application No. 63/579,505, filed on Aug. 29, 2023.

(51) Int. Cl. C12N 15/82; C12N 9/50

(52) U.S. Cl. CPC C12N 15/8261; C12N 9/63; C12N 15/8237; C12Y 304/21062

(57) ABSTRACT

The present disclosure provides novel DNA molecules and constructs encoding proteins for promoting or increasing symbiotic infection in plants. The disclosure also provides DNA molecules useful for expressing proteins in an infection-specific expression pattern. The instant disclosure also provides transgenic plants, plant cells, plant parts, seeds, and commodity products comprising the disclosed DNA molecules operably linked to heterologous transcribable polynucleotides, along with methods of their use.

### CROSS REFERENCE TO RELATED APPLICATION

This application claims the benefit of U.S. Provisional Application No. 63/579,505, filed Aug. 29, 2023, which is herein incorporated by reference in its entirety.

### INCORPORATION OF SEQUENCE LISTING

The sequence listing that is contained in the file named “AGOE013US_ST26.xml”, which is 37 KB (as measured in Microsoft Windows®) and was created on Aug. 27, 2024, is filed herewith by electronic submission and is incorporated by reference herein.

### FIELD OF THE INVENTION

The invention relates to the field of plant molecular biology and plant genetic engineering, DNA molecules useful for modulating gene expression in plants, and proteins useful for improving agronomic performance.

### BACKGROUND

Many of the world's farmers face pressure from nitrogen-deficient or phosphate-deficient soils which can result in low yield or plant death. Symbiotic bacteria can improve plant biomass under low-nitrogen conditions. Specifically, intracellular colonization of host cells by symbionts may occur between a host plant and soil-borne bacteria or fungi to produce increased growth or yield under low-nitrogen conditions. However, the genetic basis and molecular mechanisms for symbiotic nitrogen fixation have not been fully elucidated. Therefore, proteins and methods useful in promoting symbiotic infection leading are needed to provide farmers with crop plants exhibiting improved agronomic performance under nitrogen-limited conditions.

### SUMMARY OF THE INVENTION

The instant disclosure provides novel gene regulatory elements for use in plants. The present invention also provides DNA constructs comprising the regulatory elements. The instant disclosure also provides transgenic plant cells, plants, and seeds comprising the regulatory elements. The sequences may be provided operably linked to a transcribable polynucleotide molecule. In one embodiment, the transcribable polynucleotide molecule may be heterologous with respect to a regulatory sequence provided herein. A regulatory element sequence provided by the instant disclosure thus may, in particular embodiments, be defined as operably linked to a heterologous transcribable polynucleotide molecule. The present disclosure also provides methods of making and using the regulatory elements, the DNA constructs comprising the regulatory elements, and the transgenic plant cells, plants, and seeds comprising the regulatory elements operably linked to a transcribable polynucleotide molecule. The present disclosure further provides polypeptide sequences which may be heterologously expressed in plants to bring about desirable phenotypes. The instant disclosure also provides transgenic plant cells, plants, and seeds heterologously expressing or comprising these polypeptide sequences. In some embodiments, polypeptide sequences disclosed herein may be overexpressed in plants. The present disclosure also provides methods of making and using the disclosed polypeptide sequences, DNA constructs for expressing the polypeptide sequences, and transgenic plant cells, plants, and seeds comprising the polypeptide sequences. Thus, in one aspect, the instant disclosure provides a recombinant DNA molecule comprising a heterologous promoter operably linked to a polynucleotide segment encoding a subtilase (Sbt) protein or fragment thereof, wherein: a) said protein comprises an amino acid sequence of SEQ ID NO:3; b) said protein comprises an amino acid sequence having at least 70%, at least 75%, at least 80%, at least 85%, at least 90%, at least 95%, at least 98%, at least 99%, or about 100% sequence identity to SEQ ID NO:3; or c) said polynucleotide segment hybridizes under stringent hybridization conditions to a polynucleotide having the nucleotide sequence of SEQ ID NO: 3. In some embodiments, recombinant DNA molecules disclosed herein are expressed in a plant cell to produce an increase in symbiotic nitrogen fixation or recombinant DNA molecules disclosed herein are in operable linkage with a vector, and said vector is selected from the group consisting of a plasmid, phagemid, bacmid, cosmid, and a bacterial or yeast artificial chromosome. In certain embodiments, DNA molecules disclosed herein comprise a subtilase protein having at least 85% sequence identity to SEQ ID NO:3 and having subtilase activity. In several embodiments, DNA molecules disclosed herein are present within a host cell, wherein said host cell is selected from the group consisting of a bacterial cell and a plant cell. In some embodiments, said bacterial host cell is from a genus of bacteria selected from the group consisting of: Agrobacterium, Rhizobium, Bacillus, Brevibacillus, Escherichia, Pseudomonas, Klebsiella, Pantoea, and Erwinia. In further embodiments, said Bacillus is Bacillus cereus or Bacillus thuringiensis, said Brevibacillus is a Brevibacillus laterosperous, and said Escherichia is Escherichia coli. In certain embodiments, said plant cell is dicotyledonous or a monocotyledonous plant cell, such as a plant cell selected from the group consisting of an alfalfa, almond, Bambara groundnut, banana, barley, bean, black currant, broccoli, cabbage, blackberry, brassica, canola, carrot, cassava, castor, cauliflower, celery, chickpea, Chinese cabbage, citrus, coconut, coffee, corn, cowpea, clover, cotton, a cucurbit, cucumber, Douglas fir, eggplant, eucalyptus, flax, garlic, forage legumes, grape, hemp, hops, indigo, leek, legume trees, lentil, lettuce, Loblolly pine, lotus, lupin, millets, melons, Medicago spp., nut, oat, olive, onion, ornamental, palm, pasture grass, pea, peach, peanut, pepper, pigeon pea, pine, potato, poplar, pumpkin, pulses, Radiata pine, radish, rapeseed, raspberry, rice, rootstocks, rye, red currant, safflower, shrub, sorghum, Southern pine, soybean, spinach, squash, strawberry, sugar beet, sugarcane, sunflower, corn, sweet gum, sweet potato, switchgrass, tea, tobacco, tomato, triticale, turf grass, walnut, watermelon, wheat, and yam plant cells. In some embodiments, the present disclosure provide a plant, a plant part, or a plant cell comprising a recombinant DNA molecule provided herein. In certain embodiments, a plant, plant part, or plant cell described herein is a monocot plant or a dicot plant. For example, a plant or part thereof may be a plant or cell of a plant selected from the group consisting of an alfalfa, almond, Bambara groundnut, banana, barley, bean, black currant, broccoli, cabbage, blackberry, brassica, canola, carrot, cassava, castor, cauliflower, celery, chickpea, Chinese cabbage, citrus, coconut, coffee, corn, cowpea, clover, cotton, a cucurbit, cucumber, Douglas fir, eggplant, eucalyptus, flax, garlic, forage legumes, grape, hemp, hops, indigo, leek, legume trees, lentil, lettuce, Loblolly pine, lotus, lupin, millets, melons, Medicago spp., nut, oat, olive, onion, ornamental, palm, pasture grass, pea, peach, peanut, pepper, pigeon pea, pine, potato, poplar, pumpkin, pulses, Radiata pine, radish, rapeseed, raspberry, rice, rootstocks, rye, red currant, safflower, shrub, sorghum, Southern pine, soybean, spinach, squash, strawberry, sugar beet, sugarcane, sunflower, corn, sweet gum, sweet potato, switchgrass, tea, tobacco, tomato, triticale, turf grass, walnut, watermelon, wheat, and yam. In some embodiments, the instant disclosure provide a transgenic seed comprising a recombinant DNA molecule provided herein. Further provided are methods of producing progeny seed comprising the recombinant DNA molecule of claim 1, such as methods comprising: a) planting a first seed comprising the recombinant DNA molecule of claim 1; b) growing a plant from the seed of step a; and c) harvesting the progeny seed from the plants, wherein said harvested seed comprises said recombinant DNA molecule. Further provided are plants with improved symbiotic nitrogen fixation, wherein the cells of said plant comprise a recombinant DNA molecule disclosed herein. Methods are also provided for increasing symbiotic nitrogen fixation in a plant, said methods comprising: a) expressing a subtilase protein or fragment or variant thereof as set forth in SEQ ID NO:3 or at least 70% identical to SEQ ID NO: 3 having subtilase activity in a plant; and contacting said plant with an effective amount of one or more rhizobia bacterium, arbuscular mycorrhiza fungi, or a combination thereof. Methods disclosed herein include methods wherein: a) said rhizobia bacterium is selected from the group consisting of: Mesorhizobium loti, Sinorhizobium meliloti, Sinorhizobium fredii, Rhizobium sp. IRBG74 and NGR234, Bradyrhizobium sp.; or b) said arbuscular mycorrhiza fungi is selected from the group consisting of: Rhizophagus irregularis, Rhizophagus intraradices, Glomus mosseae, and Funneliformis mosseae. Further embodiments provide recombinant DNA molecules comprising a polynucleotide sequence selected from the group consisting of: a) a sequence with at least 70 percent sequence identity to SEQ ID NO:4; b) a sequence comprising SEQ ID NO:4; c) a fragment of a sequence having at least 85 percent sequence identity to SEQ ID NO:4, wherein the fragment has gene-regulatory activity; and d) a fragment of SEQ ID NO:4, wherein the fragment has gene-regulatory activity; wherein said sequence is operably linked to a heterologous transcribable polynucleotide molecule. In certain embodiments, the recombinant DNA molecule is active as a promoter. In other embodiments, the recombinant DNA molecule comprises a heterologous regulatory element. In further embodiments, said sequence provides expression of said heterologous transcribable polynucleotide molecule in response to an external stimulus. In some embodiments, said external stimulus is a presence of a rhizobia bacterium. Further provided are transgenic plant cells comprising a recombinant DNA molecule provided herein or a transgenic plant, plant part, or seed, comprising a recombinant DNA molecule provided herein. Progeny plants of plant comprising the recombinant DNA molecule are further provided. Methods of expressing a transcribable polynucleotide molecule are provided comprising obtaining a transgenic plant comprising a recombinant DNA molecule provided herein and cultivating the plant, wherein the transcribable polynucleotide is expressed. Constructs comprising at least one copy of a DNA molecule provided herein and an operably linked transcribable gene of agronomic interest are further provided, for example a construct wherein the construct comprises in the 5′-3′ direction: (a) the at least one copy of said DNA molecule; (b) the operably linked transcribable gene of agronomic interest; and (c) a gene termination sequence. In some embodiments, such constructs may comprise a transcribable gene of agronomic interest that comprises an open reading frame encoding a polypeptide. Methods of expressing a gene of agronomic interest in a plant or plant cell, the methods comprising incorporating into a plant cell a construct comprising a DNA molecule disclosed herein operably linked to a transcribable gene of agronomic interest, wherein the DNA molecule is capable of driving the expression of the operably linked gene of agronomic interest in the plant cell. Such methods may further comprise regenerating a transformed plant from said plant cell, such as a plant cell stably transformed with said construct. Methods of producing a transgenic plant cell comprising introducing a DNA molecule provided herein into a plant cell are further provided. In certain methods, introducing said DNA molecule into said plant cell comprises transformation. Some methods further comprise regenerating a transgenic plant from said plant cell. In certain methods, introducing said DNA molecule into said plant cell comprises crossing a transgenic plant provided herein with another plant to produce a progeny plant comprising said plant cell. Throughout this specification and the claims, unless the context requires otherwise, the word “comprise” and its variations, such as “comprises” and “comprising,” will be understood to imply the inclusion of a stated composition, step, and/or value, or group thereof, but not the exclusion of any other composition, step, and/or value, or group thereof.

### BRIEF DESCRIPTION OF THE FIGURES

FIG. 1 shows a phylogenic tree of representative subtilases in plants.

FIG. 2 shows the Mt12a gene (upper panel) and Mt12a protein (lower panel) structure with the position of the transposon insertion of two different mutant alleles indicated (12a-1 and 12a-2).

FIG. 3 shows a plant comprising a wildtype Mt12a compared with plants comprising mutant Mt12a-1 and Mt12a-2 alleles. Plants of 12a mutants exhibited a nitrogen-defective growth phenotype. Bar=1 cm.

FIG. 4 shows the nodule phenotype of wild type (R108) and Mt12a-1 and Mt12a-2 mutants 18 days after inoculation with S. meliloti. Bar=1 mm.

FIG. 5 shows the nodule numbers of wild type (R108) and Mt12a-1 and Mt12a-2 mutants 18 days after inoculation. n=18, 18 and 17, respectively.

FIG. 6 shows infection phenotypes of wildtype (R108) and Mt12a mutant plants. Abnormal infection thread (IT) development is marked with arrow heads. Bar=50 um.

FIG. 7 shows the number and percentage of aborted ITs in wildtype (R108) and Mt12a-1 (NF1441) and Mt12a-2 (NF18072) mutant plants. Two-tailed Student's t-test, ***p<0.001, ****p<0.0001, n=18, 18 and 17, respectively.

FIG. 8 shows phenotypical analyses of Mt12a mutants. (Panel A) Plants of Mt12a mutants exhibited a nitrogen-starvation growth phenotype as indicated by a stunted growth, chlorotic leaves and anthocyanin production. (Panel B) Numbers of Wild Type (R108, WT) and Mt12a mutant nodules 21 days post inoculation (dpi) with S. meliloti Sm2011. n=28, 22 and 24, respectively. (Panels C-E) Representative images of nodules developed on WT, Mt12a-1 and Mt12a-2. Bars=1 cm (A) and 1 mm (C-E).

FIG. 9 shows modified zonation in the nodules of plants comprising Mt12a-1 and Mt12a-2 mutant alleles compared with plants comprising wildtype Mt12a (R108). Sections of nodules from wildtype and Mt12a-1 and Mt12a-2 mutant plants are shown at 18 days post-inoculation with S. meliloti. Nodules were embedded in Technovit and stained with Toluidine blue after sectioning. The nodules contain either no nitrogen-fixing cells or exhibited modified zonation. The senescence zones (IV) are marked by dotted lines. Bars=200 μm.

FIG. 10 shows genetic complementation of Mt12a mutants. Representative images of nodules developed on complemented roots harvested at 21 dpi. Bars=1 mm.

FIG. 11 shows decreased nitrogenase activity in mt12 mutants. Nitrogenase activity was assessed using an acetylene reduction assay as is displayed as activity measured from all nodules per plant after 21 dpi.

FIG. 12 shows that mt12a is involved in symbiosome development. (Panels A and B) Representative images showing differences between symbiosomes isolated from WT and Mt12a-2 nodules. Purified symbiosomes were stained with PI (Propidium iodide, magenta). (Panel C) Symbiosomes of mt12a-2 were shorter compared to WT in a quantification assay. n=89 and 98, respectively. Bars=20 μm.

FIG. 13 shows a qRT-PCR analysis of Mt12a transcript levels in roots of wild type plants. Samples were collected at 0, 1, 3, 5, 7, 10, and 14 days post-inoculation (dpi) with S. meliloti. n=3, means and SE are shown.

FIG. 14 shows a qRT-PCR analysis of Mt12a transcript levels in roots of wild type and symbiosis deficient mutants. Samples were harvested at 10 dpi. n=3, means and SE are shown.

FIG. 15 shows Mt12a transcript levels in different zones of nodules obtained from Expression Atlas of Medicago truncatula.

FIG. 16 shows expression analyses of Medicago subtilase genes. (Panels A-D) ProSbt12a:GUS expression in a root hair harboring an infection thread (Panel A), infected cortical cells (Panel B), young nodule primordia (Panel C) and a mature nodule shown by semi-thin longitudinal sections (Panel D). (Panels E and F) ProSbt12b:GUS expression during nodule organogenesis (Panel E) and lateral root initiation (Panel F). Blue color indicates GUS activity and magenta color indicates staining of S. meliloti 2011. Composite roots were inoculated with rhizobia and harvested at 7 and 14 dpi. Bars=100 μm (Panels A, B and D), 200 μm (Panels C and F) and 500 μm (Panel E), respectively.

FIG. 17 shows subcellular localization of MtCG12a protein. Panels A to C show confocal images showing the MtCG12a-eGFP fusion (green) driven by ProLjUbi promoter in root hair cells harboring infection chambers (Panel A) and infection threads (Panels B and C).

FIG. 18 shows confocal images showing the MtCG12a-eGFP fusion (green) driven by ProLjUbi promoter in infected cells from nodules.

FIG. 19 shows Fluorescence Lifetime Imaging Microscopy (FLIM) images (same cells as FIG. 14) showing the localization of 12a in symbiosome structure. The composite roots were inoculated with S. meliloti mCherry (Magenta) and harvested at 21 days post-inoculation. Bars=20 μm.

FIG. 20 shows workflow for High-efficiency Undecanal-based N Termini EnRichment (HUNTER). Adapted from Demir, et al., 2022.

FIG. 21 shows that expression of MT12a is NIN-dependent. (Panel A) RealTime PCR experiments show that Mt12a expression is not induced in the nin-1 mutant upon inoculation with S. meliloti. (Panel B) In line with data shown in (Panel A), the promoter of MT12a is not activated above background levels in the nin-1 mutant. GUS staining was done 5 dpi with S. meliloti. Scale bars indicate 50 mm. (Panel C). Transactivation assay done in N. benthamiana leaves shows activation of the ProSbt12a:GUS reporter upon co-expression with NIN but not with NF-YA1.

### BRIEF DESCRIPTION OF THE SEQUENCES

SEQ ID NO:1 is the Mt12a genomic sequence.

SEQ ID NO:2 is the Mt12a mRNA sequence.

SEQ ID NO:3 is the Mt12a coding sequence.

SEQ ID NO:4 is the Mt12a promoter.

SEQ ID NO: 5 is the amino acid sequence encoded by SEQ ID NO:3.

SEQ ID NO:6 is the Mt12a nucleotide sequence in the Mt12a-1 mutant.

SEQ ID NO:7 is the Mt12a amino acid sequence in the Mt12a-1 mutant.

SEQ ID NO:8 is the Mt12a nucleotide sequence in the Mt12a-2 mutant.

SEQ ID NO:9 is the Mt12a amino acid sequence in the Mt12a-2 mutant.

### DETAILED DESCRIPTION OF THE INVENTION

Improving crop yield from agriculturally significant plants has become increasingly important. In addition to the growing need for agricultural products to feed, clothe, and provide energy for a growing human population, climate-related effects and pressures are predicted to reduce the amount of arable land available for farming. These factors have led to grim forecasts of food security, particularly in the absence of major improvements in plant biotechnology and agronomic practices. In light of these pressures, environmentally sustainable improvements in technology, agricultural techniques, and pest management are vital tools to expand crop production on the limited amount of arable land available for farming.

Many of the world's farmers also face pressure from nitrogen-deficient or phosphate-deficient soils which can result in low yield or plant death. Symbiotic bacteria can improve plant biomass under low-nitrogen conditions. Specifically, intracellular colonization of host cells by symbionts may occur between a host plant and soil-borne bacteria or fungi to produce increased growth or yield under low-nitrogen conditions. The instant disclosure therefore provides proteins involved in promoting symbiotic nitrogen fixation, together with regulatory elements for advantageous spatial and temporal expression of such proteins.

Subtilases constitute a large group of serine proteases and regulate both general protein turnover and specific precursor processing. In plants, subtilases are involved in many aspects including plant development and interaction with the environment. However, genetic evidence of the activity of subtilases is widely missing and molecular mechanisms remain elusive. The present inventors have identified and characterized the Medicago truncatula subtilase gene, Mt12a, and shown that it is required for successful symbiotic nitrogen fixation. Mt12a is induced following rhizobial inoculation and displays an infection-specific expression pattern. The Mt12a protein localizes to the extracellular space of rhizobium-infected cells.

In certain embodiments, the instant disclosure therefore provides recombinant DNA molecules as well as plants, plant cells, plant parts, or seeds comprising recombinant DNA molecules encoding Mt12 proteins or fragments or variants thereof. These plants, plant cells, plant parts, or seeds may comprise recombinant DNA molecules comprising SEQ ID NO:3, or fragments or variants thereof.

The instant disclosure further provides regulatory polynucleotide molecules capable of providing unique spatial and temporal expression of operably linked proteins. In certain embodiments, regulator polynucleotide molecules provided include SEQ ID NO:4, or fragments or variants thereof. These polynucleotide molecules are, for instance, capable of affecting the expression of an operably linked transcribable polynucleotide molecule in plant tissues, and selectively regulating gene expression or activity of an encoded gene product in transgenic plants.

### Symbiotic Bacteria

The present invention provides DNA molecules encoding proteins that when expressed in a plant may promote symbiotic nitrogen fixation or express a transcribable polynucleotide molecule in response to symbiotic infection. Rhizobia are bacteria found in soil that infect the roots of legumes and colonize root nodules which are involved in nitrogen utilization. As used herein, “rhizobia” refers to any diazotrophic bacteria that fix atmospheric nitrogen inside plants roots.

Symbiotic bacteria can be used with plants comprising the recombinant DNA molecules described herein to produce improved agronomic effects including improved plant growth or increased yield or biomass under reduced nitrogen conditions. Symbiotic bacteria useful with the disclosed plants include, but are not limited to, Mesorhizobium loti, Sinorhizobium meliloti, Sinorhizobium fredii, Rhizobium sp. IRBG74 and NGR234, Bradyrhizobium sp.

### Subtilase Genes

Subtilases are a family of serine proteases that can be found in all kingdoms of life and perform a variety of physiological functions. As described herein, subtilases play a role in mutualistic symbiosis and host-pathogen interactions in angiosperm lineages, and in stress responses and adaptation mechanisms in plants. They are also involved in many developmental processes in land plants, including embryogenesis, organogenesis, senescence, and programmed cell death.
The first subtilase gene family to be described belonged to Lycopersicon esculentum (tomato) and consisted of 15 members (Meichtry et al., 1999). After this work, subtilase gene families were also characterized in Arabidopsis thaliana, presenting 56 known genes (Rautengarten et al., 2005), in Oryza sativa (rice) with 63 genes (Tripathi and Sowdhamini, 2006), in moss Physcomitrella patens with 23 genes, in Populus trichocarpa with 90 genes (Schaller et al., 2012) and, more recently, in Vitis vinifera and Solanum tuberosum (potato), both with 82 known genes (Figueiredo et al., 2016; Norero et al., 2016).
Experiments to determine subtilase structure and function in plants as well as experiments to determine if a plant comprises a subtilase gene could be carried out by a person of ordinary skill in the art given the disclosure herein. Functional orthologs and homologs of the subtilase gene, such as Mt12a, can be identified through comparative structural analysis of homology.

TABLE 1
Examples of known subtilases.

| | % Identity to | |
|---|---|---|
| Scientific Name for Sbt Species | SEQ ID NO: 5 | Gene Accession |
| Medicago truncatula | 100 | ABD28577.1 |
| Medicago truncatula | 83.72 | XP_003624105.1 |
| Vicia villosa | 82.54 | XP_058739949.1 |
| Lathyrus sativus | 81.63 | CAK8542465.1 |
| Pisum sativum | 81.89 | XP_050914398.1 |
| Trifolium pratense | 82.22 | XP_045793036.1 |
| Trifolium repens | 81.22 | WJX82891.1 |
| Pisum sativum | 80.52 | XP_050912056.1 |
| Vicia villosa | 80.87 | XP_058754964.1 |
| Trifolium pratense | 81.05 | CAJ2633375.1 |
| Vicia villosa | 79.79 | XP_058737421.1 |
| Medicago truncatula | 100 | XP_003624104.3 |
| Medicago truncatula | 100 | RHN47059.1 |
| Trifolium pratense | 80.49 | CAJ2670812.1 |
| Trifolium repens | 79.97 | WJX77465.1 |
| Trifolium pratense | 80.36 | XP_045797393.1 |
| Trifolium repens | 81.68 | WJX82895.1 |
| Vicia villosa | 79.28 | XP_058754975.1 |
| Trifolium pratense | 79.97 | XP_045798224.1 |
| Trifolium repens | 81.06 | KAK2415641.1 |
| Trifolium repens | 80.93 | WJX77474.1 |
| Vicia faba | 79.59 | CAI8613220.1 |
| Lathyrus sativus | 79.28 | CAK8542468.1 |
| Trifolium repens | 81.87 | KAK2361881.1 |
| Trifolium repens | 82.08 | KAK2361884.1 |
| Trifolium repens | 80.7 | KAK2415631.1 |
| Pisum sativum | 78.32 | XP_050912057.1 |
| Vicia villosa | 78.56 | XP_058754965.1 |
| Trifolium repens | 80.8 | KAK2361818.1 |
| Gastrolobium bilobum | 75.94 | XP_061360169.1 |
| Vicia faba | 83.07 | CAI8613218.1 |
| Vicia villosa | 75.03 | XP_058754976.1 |
| Trifolium pratense | 83.87 | PNY17185.1 |
| Trifolium pratense | 77.66 | PNY01530.1 |
| Trifolium repens | 82.46 | WJX82890.1 |
| Trifolium repens | 82.53 | WJX82896.1 |
| Trifolium repens | 81.54 | WJX77475.1 |
| Trifolium repens | 81.49 | WJX77464.1 |
| Spatholobus suberectus | 72.15 | TKY65378.1 |
| Cicer arietinum | 73.64 | XP_004492811.1 |
| Canavalia gladiata | 71.63 | KAK7340331.1 |
| Glycine max | 71.58 | KAG5095749.1 |
| Vigna unguiculata | 71.8 | QCE12398.1 |
| Glycine soja | 71.58 | XP_028212579.1 |
| Vigna unguiculata | 71.89 | XP_027930022.1 |
| Glycine max | 71.45 | KAG4922494.1 |
| Glycine max | 71.32 | KAG4937253.1 |
| Trifolium repens | 81.45 | WJX82846.1 |
| Canavalia gladiata | 70.16 | KAK7344117.1 |
| Vigna radiata var. radiata | 72.14 | XP_014496471.1 |
| Glycine max | 71.32 | XP_003552427.1 |
| Psophocarpus tetragonolobus | 71.13 | KAK7394694.1 |
| Spatholobus suberectus | 71.15 | TKY52050.1 |
| Phaseolus coccineus | 71.54 | KAK7374349.1 |
| Vigna umbellata | 71.08 | XP_047164487.1 |
| Abrus precatorius | 70.38 | XP_027364570.1 |
| Cajanus cajan | 70.5 | XP_020231871.1 |
| Vigna angularis | 71.21 | KAG2404249.1 |
| Psophocarpus tetragonolobus | 69.21 | KAK7376341.1 |
| Vigna unguiculata | 70.63 | QCE12400.1 |
| Vigna angularis | 71.21 | XP_017418292.1 |
| Sphenostylis stenocarpa | 70.75 | CAJ1821881.1 |
| Sesbania bispinosa | 70.67 | KAJ1409309.1 |
| Glycine soja | 71.34 | XP_028183868.1 |
| Phaseolus vulgaris | 71.02 | XP_007139760.1 |
| Vigna mungo | 72.11 | WVZ05976.1 |
| Phaseolus coccineus | 69.21 | KAK7356772.1 |
| Glycine soja | 71.34 | KAG4983578.1 |
| Glycine max | 71.21 | XP_003535388.1 |
| Phaseolus coccineus | 70.38 | KAK7374346.1 |
| Abrus precatorius | 70.45 | XP_027349363.1 |
| Vigna angularis var. angularis | 70.32 | BAT83553.1 |
| Vigna angularis | 69.47 | XP_017410930.1 |
| Vigna angularis | 70.06 | KOM37046.1 |
| Canavalia gladiata | 71.43 | KAK7340330.1 |
| Sphenostylis stenocarpa | 70.08 | CAJ1976316.1 |
| Phaseolus vulgaris | 68.43 | XP_007143987.1 |
| Vigna mungo | 70.3 | WVZ05975.1 |
| Cajanus cajan | 71.47 | KYP74574.1 |
| Vigna radiata var. radiata | 69.67 | XP_014511851.1 |
| Vigna unguiculata | 68.82 | XP_027937876.1 |
| Vigna angularis | 69.82 | KAG2402180.1 |
| Lupinus albus | 70.03 | KAF1880362.1 |
| Vigna umbellata | 69.85 | XP_047164459.1 |
| Vigna unguiculata | 68.82 | QCE07495.1 |
| Clitoria ternatea | 69.51 | KAK7278272.1 |
| Crotalaria pallida | 70.58 | KAK7267819.1 |
| Lupinus luteus | 70.21 | CAL0316638.1 |
| Vigna angularis | 69.46 | XP_017416420.1 |
| Cajanus cajan | 71.41 | XP_020234802.1 |
| Mucuna pruriens | 69.33 | RDY01051.1 |
| Vigna umbellata | 69.21 | XP_047159435.1 |
| Vigna mungo | 70.13 | WVZ05973.1 |
| Glycine max | 70.22 | KAH1191937.1 |
| Sphenostylis stenocarpa | 68.94 | CAJ1821712.1 |
| Gastrolobium bilobum | 69.61 | XP_061342603.1 |
| Glycine max | 69.13 | KAG4908475.1 |
| Vigna radiata var. radiata | 70.17 | XP_014497157.2 |
| Arachis duranensis | 67.38 | XP_020996651.1 |
| Arachis stenosperma | 66.75 | XP_057757354.1 |

TABLE 2
Examples of known subtilases.

| | % Identity to | Nucleotide |
|---|---|---|
| Scientific Name for Sbt Species | SEQ ID NO: 3 | Accession |
| Medicago truncatula | 100 | XM_003624056.4 |
| Medicago truncatula | 100 | AC148994.3 |
| Medicago truncatula | 89.6 | XM_003624057.4 |
| Vicia villosa | 87.69 | XM_058883966.1 |
| Pisum sativum | 87.58 | XM_051058441.1 |
| Trifolium pratense | 87.16 | XM_045937080.1 |
| Vicia villosa | 86.64 | XM_058898981.1 |
| Pisum sativum | 86.69 | XM_051056099.1 |
| Trifolium pratense | 86.15 | XM_045941437.1 |
| Trifolium pratense | 86.11 | XM_045942268.1 |
| Vicia villosa | 85.79 | XM_058881438.1 |
| Vicia villosa | 85.45 | XM_058898992.1 |
| Vicia villosa | 84.69 | XM_058898993.1 |
| Gastrolobium bilobum | 81.7 | XM_061504185.1 |
| Vicia villosa | 84.49 | XM_058898994.1 |
| Pisum sativum | 86.23 | XM_051056101.1 |
| Glycine soja | 79.77 | XM_028356778.1 |
| Glycine max | 79.59 | XM_003552379.5 |
| Abrus precatorius | 79.93 | XM_027493562.1 |
| Phaseolus vulgaris | 78.79 | XM_007139698.1 |
| Vigna radiata var. radiata | 79.66 | XM_014640985.2 |
| Vigna angularis | 79.24 | XM_017562803.2 |
| Cicer arietinum | 83.37 | XM_004492754.3 |
| Vigna radiata var. radiata | 78.91 | XM_014641671.2 |
| Vigna umbellata | 79.14 | XM_047308531.1 |
| Vigna umbellata | 78.87 | XM_047308503.1 |
| Vigna angularis | 78.78 | XM_017560931.2 |
| Vicia villosa | 86.21 | XM_058898982.1 |
| Pisum sativum | 85.51 | XM_051056100.1 |
| Glycine soja | 76.54 | XM_028365757.1 |
| Medicago truncatula | 75.2 | XM_003590818.4 |
| Phaseolus vulgaris | 80.25 | XM_007139701.1 |
| Phaseolus vulgaris | 78.04 | KM061210.1 |
| Pyrus x bretschneideri | 76.38 | XM_009344736.3 |
| Malus domestica | 75.1 | XM_008372690.3 |
| Malus domestica | 74.81 | XM_017331949.2 |
| Malus sylvestris | 74.81 | XM_050288652.1 |
| Malus sylvestris | 74.68 | XM_050288651.1 |
| Manihot esculenta | 74.68 | XM_021745845.2 |
| Ricinus communis | 75.16 | XM_002510148.4 |
| Durio zibethinus | 73.87 | XM_022910222.1 |
| Hevea brasiliensis | 72.18 | XM_058129589.1 |
| Mangifera indica | 89.06 | XM_044631035.1 |
| Populus alba | 84.51 | XM_035041677.1 |
| Populus nigra | 84.51 | XM_062104528.1 |
| Populus euphratica | 84.51 | XM_011031327.1 |
| Cucurbita maxima | 79.31 | XM_023111420.1 |
| Corylus avellana | 78.12 | XM_059606778.1 |
| Solanum tuberosum | 88.24 | XM_006355558.2 |
| Populus trichocarpa | 81.69 | XM_006374850.3 |
| Solanum stenotomum | 88.24 | XM_049527463.1 |
| Solanum verrucosum | 88.24 | XM_049515315.1 |
| Manihot esculenta | 89.36 | XM_021773767.2 |
| Cucurbita pepo subsp. pepo | 83.61 | XM_023677051.1 |
| Cucurbita maxima | 83.61 | XM_023139497.1 |
| Cucurbita moschata | 83.61 | XM_023075158.1 |
| Tripterygium wilfordii | 86.54 | XM_038858629.1 |
| Nicotiana tomentosiformis | 92.5 | XM_009605978.3 |
| Lycium barbarum | 92.5 | XM_060335872.1 |
| Lycium ferocissimum | 92.5 | XM_059433816.1 |
| Nicotiana tabacum | 92.5 | XM_016607139.1 |
| Mercurialis annua | 96.97 | XM_050349910.2 |
| Ricinus communis | 96.97 | XM_002526491.4 |

TABLE 3
Examples of known subtilases.

| Scientific Name for Sbt Species | Nucleotide Accession |
|---|---|
| Oryza sativa Japonica Group | NC_089035.1 |
| Arabidopsis lyrata subsp. lyrata | NW_003302552.1 |
| Nicotiana tabacum | NW_015790789.1 |
| Cicer arietinum | NC_021164.1 |
| Cicer arietinum | NC_021160.1 |
| Cicer arietinum | NC_021161.1 |
| Cicer arietinum | NC_021164.1 |
| Cicer arietinum | NC_021165.1 |
| Cicer arietinum | NC_021162.1 |
| Cicer arietinum | NC_021162.1 |
| Nicotiana tabacum | NW_015931416.1 |
| Medicago truncatula | NC_053046.1 |
| Lotus japonicus | NC_080044.1 |
| Lotus japonicus | NC_080042.1 |
| Lotus japonicus | NC_080042.1 |
| Lotus japonicus | NC_080043.1 |
| Arabidopsis lyrata subsp. lyrata | NW_003302555.1 |

### DNA Molecules

As used herein, the term “DNA” or “DNA molecule” refers to a double-stranded DNA molecule of genomic or synthetic origin, i.e., a polymer of deoxyribonucleotide bases or a polynucleotide molecule, read from the 5′ (upstream) end to the 3′ (downstream) end. As used herein, the term “DNA sequence” refers to the nucleotide sequence of a DNA molecule. The nomenclature used herein corresponds to that of by Title 37 of the United States Code of Federal Regulations § 1.822, and set forth in the tables in WIPO Standard ST.25 (1998), Appendix 2, Tables 1 and 3.

As used herein, a “recombinant DNA molecule” is a DNA molecule comprising a combination of DNA molecules that would not naturally occur together without human intervention. For instance, a recombinant DNA molecule may be a DNA molecule that is comprised of at least two DNA molecules heterologous with respect to each other, a DNA molecule that comprises a DNA sequence that deviates from DNA sequences that exist in nature, a DNA molecule that comprises a synthetic DNA sequence or a DNA molecule that has been incorporated into a host cell's DNA by genetic transformation or gene editing.

As used herein, the term “isolated DNA molecule” refers to a DNA molecule at least partially separated from other molecules normally associated with it in its native or natural state. In one embodiment, the term “isolated” refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

A polynucleotide or polypeptide provided herein may further include two or molecules which are heterologous with respect to one another. As used herein, the term “heterologous” refers to the combination of two or more polynucleotide molecules or two or more polypeptide molecules when such a combination is not normally found in nature. For example, the two molecules may be derived from different species and/or the two molecules may be derived from different genes, e.g., different genes from the same species or the same genes from different species. In some examples, a promoter is heterologous with respect to an operably linked transcribable polynucleotide molecule if such a combination is not normally found in nature, i.e., that transcribable polynucleotide molecule is not naturally occurring operably linked in combination with that promoter molecule. Additionally, the two molecules can be derived from isolated locations in the same gene, wherein such a combination of molecules is not normally found in nature.

The terms “heterologous DNA sequence”, “exogenous DNA segment”, or “heterologous nucleic acid”, “transgene”, “exogenous polynucleotide” as used herein, each refers to a sequence that originates from a source foreign (e.g., non-native) to the particular host cell or, if from the same source or species, is modified from its original form and/or genetic locus; is heterologous to a host cell at least with respect to its location in the genome; the promoter is not the native promoter for the operably linked polynucleotide, and/or is artificially incorporated into a host cell's genome in the current or any prior generation of the cell.

Any number of methods well known to those skilled in the art can be used to isolate and manipulate a DNA molecule, or fragment thereof, disclosed in the present invention. For example, PCR (polymerase chain reaction) technology can be used to amplify a particular starting DNA molecule and/or to produce variants of the original molecule. DNA molecules, or fragment thereof, can also be obtained by other techniques such as by directly synthesizing the fragment by chemical means, as is commonly practiced by using an automated oligonucleotide synthesizer.

As used herein, the term “percent sequence identity,” “percent identity,” or “% sequence identity” refers to the percentage of identical nucleotides or amino acids in a linear polynucleotide or polypeptide sequence of a reference (e.g., “query”) sequence (or its complementary strand) as compared to a test (e.g., “subject”) sequence (or its complementary strand) when the two sequences are optimally aligned. An optimal sequence alignment is created by manually aligning two sequences, e.g., a reference sequence and another sequence, to maximize the number of nucleotide matches in the sequence alignment with appropriate internal nucleotide insertions, deletions, or gaps. Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, CA), MEGAlign (DNAStar, Inc., 1228 S. Park St., Madison, Wis. 53715), and MUSCLE (version 3.6) (RC Edgar, Nucleic Acids Research (2004) 32(5):1792-1797) with default parameters. An “identity fraction” for aligned segments of a test sequence and a reference sequence is the number of identical components which are shared by the two aligned sequences divided by the total number of components in the reference sequence segment, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence. As used herein, the term “sequence identity” refers to the extent to which two optimally aligned polynucleotide sequences or two optimally aligned polypeptide sequences are identical. As used herein, the term “reference sequence,” for example, may refer to a sequence provided as the polynucleotide sequences of any of SEQ ID NOs:1-4, 6, and 8 or the polypeptide sequences of any of SEQ ID NOs:5, 7, and 9.

Thus, one embodiment of the invention is a recombinant DNA molecule comprising a sequence that when optimally aligned to a reference sequence, provided herein as the polynucleotide sequences of SEQ ID NO:1-4, 6, or 8 has at least about 85 percent identity, at least about 90 percent identity, at least about 95 percent identity, at least about 96 percent identity, at least about 97 percent identity, at least about 98 percent identity, or at least about 99 percent identity to the sequence of SEQ ID NO:1-4, 6, or 8. In particular embodiments such sequences may be defined as having the activity of the reference sequence, for example the activity of any of SEQ ID NO:1-4, 6, or 8.

Similarly, another embodiment of the invention is a polypeptide molecule comprising a sequence that when optimally aligned to a reference sequence, provided herein as the polypeptide sequences of SEQ ID NOs:5, 7, and 9 has at least about 85 percent identity, at least about 90 percent identity, at least about 95 percent identity, at least about 96 percent identity, at least about 97 percent identity, at least about 98 percent identity, or at least about 99 percent identity to the sequence of SEQ ID NO:5, 7, or 9. In particular embodiments such sequences may be defined as having the activity of the reference sequence, for example the activity of any of SEQ ID NOs: 5, 7, and 9.

Also provided are fragments of polynucleotide sequences provided herein, for example fragments of a polynucleotide sequence selected from SEQ ID NO:1-4, 6, or 8. In specific embodiments, fragments of a polynucleotide sequences are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, at least about 1000, at least about 1250, at least about 1500, at least about 1750, or at least about 2000 contiguous nucleotides, or longer, of a DNA molecule of any of SEQ ID NO:1-4, 6, or 8. Methods for producing such fragments from a starting molecule are well known in the art. Fragments of a polynucleotide sequence provided herein may comprise the activity of the base sequence.

Disclosed sequences may hybridize specifically to a target DNA sequence under stringent hybridization conditions. In certain embodiments, polynucleotides disclosed herein may hybridize under stringent hybridization conditions to a polynucleotide having the nucleotide sequence of SEQ ID NO:1-4, 6, or 8. Stringent hybridization conditions are known in the art and described in, for example, MR Green and J Sambrook, Molecular cloning: a laboratory manual, 4^{th} Edition, Cold Spring Harbor Laboratory Press, Cold Spring Harbor, N.Y. (2012). As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, two molecules exhibit “complete complementarity” if when aligned every nucleotide of the first molecule is complementary to every nucleotide of the second molecule. Two molecules are “minimally complementary” if they can hybridize to one another with sufficient stability to permit them to remain annealed to one another under at least conventional “low-stringency” conditions. Similarly, the molecules are “complementary” if they can hybridize to one another with sufficient stability to permit them to remain annealed to one another under conventional “high-stringency” conditions. Departures from complete complementarity are therefore permissible, as long as such departures do not completely preclude the capacity of the molecules to form a double-stranded structure.

Appropriate stringency conditions that promote DNA hybridization, for example, 6.0×sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

Recombinant polynucleotide sequences encoding fragments of polypeptide sequences provided herein are further envisioned, including polynucleotide sequences encoding fragments of a polypeptide sequence selected from SEQ ID NOs: 5, 7, and 9. In specific embodiments, fragments of a polypeptide are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous amino acids, or longer, of a polypeptide molecule selected from SEQ ID NOs: 5, 7, and 9. Methods for producing such fragments from a starting molecule are well known in the art. Fragments of a polynucleotide sequence provided herein may maintain the activity of the base sequence.

### Transcribable Polynucleotide Molecules

Recombinant DNA molecules provided herein include transcribable polynucleotide molecules or sequences encoding useful polypeptide sequences. In certain examples, transcribable polynucleotide molecules include sequences encoding Mt12a, or Mt12a-related polypeptides. Transcribable polynucleotides provided herein include SEQ ID NOs:1-3, or polynucleotide sequences encoding any of SEQ ID NOs:5, 7, or 9, or fragments or variants thereof.

As used herein, the term “transcribable polynucleotide molecule” refers to any DNA molecule capable of being transcribed into a RNA molecule, including, but not limited to, those having protein coding sequences and those producing RNA molecules having sequences useful for gene suppression.

The type of DNA molecule can include, but is not limited to, a DNA molecule from the same plant, a DNA molecule from another plant, a DNA molecule from a different organism, or a synthetic DNA molecule, such as a DNA molecule containing an antisense message of a gene, or a DNA molecule encoding an artificial, synthetic, or otherwise modified version of a transgene. Exemplary transcribable DNA molecules for incorporation into constructs of the invention can include, e.g., DNA molecules or genes from a species other than the species into which the DNA molecule is incorporated or genes that originate from, or are present in, the same species, but are incorporated into recipient cells by genetic engineering methods rather than classical breeding techniques.

With respect to polypeptide sequences, the term “variant” as used herein refers to a second polypeptide sequence that is in composition similar, but not identical to, a first polypeptide sequence and yet the second polypeptide sequence still maintains the general functionality, i.e., same or similar activity, of the first polypeptide sequence. A variant may be a shorter or truncated version of the first polypeptide sequence and/or an altered version of the sequence of the first polypeptide sequence, such as one with different amino acid deletions, substitutions, and/or insertions. Variants having a percent identity to a sequence disclosed herein may have the same activity as the base sequence. For example, the transcribable polynucleotide molecule can encode a protein or variant of a protein or fragment of a protein that is functionally defined to maintain activity in transgenic host cells including plant cells, plant parts, explants, and whole plants.

Similarly, with respect to polynucleotide sequences, the term “variant” as used herein refers to a second polynucleotide sequence that is in composition similar, but not identical to, a first polynucleotide sequence and yet the second polynucleotide sequence still maintains the general functionality, i.e., same or similar activity, of the first polynucleotide sequence. A variant may be a shorter or truncated version of the first polynucleotide sequence and/or an altered version of the sequence of the first polynucleotide sequence, such as one with different nucleotide deletions, substitutions, and/or insertions. Variants having a percent identity to a sequence disclosed herein may have the same activity as the base sequence. For example, variant polynucleotides may encode the same or a similar protein sequence or have the same or similar gene regulatory activity as the base sequence.

The transcribable polynucleotide molecule may generally be any DNA molecule for which expression of a RNA transcript is desired. Such expression of an RNA transcript may result in translation of the resulting mRNA molecule and thus protein expression. Alternatively, for example, a transcribable polynucleotide molecule may be designed to ultimately cause decreased expression of a specific gene or protein. In one embodiment, this may be accomplished by using a transcribable polynucleotide molecule that is oriented in the antisense direction. One of ordinary skill in the art is familiar with using such antisense technology. Briefly, as the antisense transcribable polynucleotide molecule is transcribed, the RNA product hybridizes to and sequesters a complementary RNA molecule inside the cell. This duplex RNA molecule cannot be translated into a protein by the cell's translational machinery and is degraded in the cell. Any gene may be negatively regulated in this manner.

Thus, one embodiment of the invention is a regulatory element of the present invention, such as those provided as SEQ ID NO: 4 or variants and fragments thereof, operably linked to a transcribable polynucleotide molecule so as to modulate transcription of the transcribable polynucleotide molecule at a desired level or in a desired pattern when the construct is integrated in the genome of a plant cell. In one embodiment, the transcribable polynucleotide molecule comprises a protein-coding region of a gene, and the promoter affects the transcription of an RNA molecule that is translated and expressed as a protein product.

In another embodiment, the transcribable polynucleotide molecule comprises an antisense region of a gene, and the promoter affects the transcription of an antisense RNA molecule, double stranded RNA or other similar inhibitory RNA molecule in order to inhibit expression of a specific RNA molecule of interest in a target host cell.

As used herein, “modulation” of expression refers to the process of effecting either overexpression or suppression of a polynucleotide or a protein.

As used here, the term “overexpression” as used herein refers to an increased expression level of a polynucleotide or a protein in a plant, plant cell or plant tissue, compared to expression in a wild-type plant, cell, or tissue, at any developmental or temporal stage for the gene. Overexpression can take place in plant cells normally lacking expression of polypeptides functionally equivalent or identical to the present polypeptides. Overexpression can also occur in plant cells where endogenous expression of the present polypeptides or functionally equivalent molecules normally occurs, but such normal expression is at a lower level. Overexpression thus results in a greater than normal production, or “overproduction” of the polypeptide in the plant, cell, or tissue.

Overexpression can be achieved using numerous approaches. In one embodiment, overexpression can be achieved by placing the DNA sequence encoding one or more polynucleotides or polypeptides under the control of a promoter, examples of which include but are not limited to endogenous promoters, homologous promoters, heterologous promoters, inducible promoters, development specific promoters, and tissue specific promoters. In one exemplary embodiment, the promoter is a constitutive promoter, for example, the cauliflower mosaic virus 35S promoter and other constitutive promoters known in the art. Thus, depending on the promoter used, overexpression can occur throughout a plant, in specific tissues of the plant, in specific stages of development of the plant, or in the presence or absence of different inducing or inducible agents, such as hormones or environmental signals.

In certain embodiments, the expression or overexpression of a transcribable polynucleotide molecule encoding a protein as disclosed herein can affect an enhanced trait or altered phenotype directly or indirectly. In the latter case it may do so, for example, by promoting symbiotic infection process. In an exemplary embodiment, the protein produced from the transcribable polynucleotide molecule can enhance or increase symbiotic nitrogen fixation in a plant.

Transcribable polynucleotide molecules may be genes of agronomic interest. As used herein, the term “gene of agronomic interest” refers to a transcribable polynucleotide molecule that when expressed in a particular plant tissue, cell, or cell type confers a desirable characteristic, such as associated with plant morphology, physiology, growth, development, yield, product, nutritional profile, disease or pest resistance, and/or environmental or chemical tolerance. Genes of agronomic interest include, but are not limited to, those encoding a yield protein, a stress resistance protein, a developmental control protein, a tissue differentiation protein, a meristem protein, an environmentally responsive protein, a senescence protein, a hormone responsive protein, an abscission protein, a source protein, a sink protein, a flower control protein, a seed protein, an herbicide resistance protein, a disease resistance protein, a fatty acid biosynthetic enzyme, a tocopherol biosynthetic enzyme, an amino acid biosynthetic enzyme, a pesticidal protein, or any other agent such as an antisense or RNAi molecule targeting a particular gene for suppression. The product of a gene of agronomic interest may act within the plant in order to cause an effect upon the plant physiology or metabolism.

In certain examples provided herein, a “gene of agronomic interest” also refers to transcribable polynucleotide molecules involved in symbiotic infection. For example, such genes of agronomic interest may include Nodule Inception (NIN) factors, Nod factor receptors, Nodule Pectate Lyase (NPL), Symbiotic Remorin 1 (SYMREM1), Rhizobium-directed polar growth (RPG), Interacting Protein of DMI3 (IPD3), and CYCLOPS.

In some embodiments of the invention, a promoter is incorporated into a construct such that the promoter is operably linked to a transcribable polynucleotide molecule that encodes a subtilase or subtilase-related protein, including any of SEQ ID NOs:5, 7, and 9, or fragments or variants thereof. The expression of the transcribable polynucleotide molecule is desirable in order to confer an agronomically beneficial trait, including but not limited to improved capacity for symbiotic infection. An agronomically beneficial trait may also be, for example, improved or modified yield, plant growth and development, biomass, grain characteristics, hybrid seed production utility, fiber production, sterility, abiotic stress tolerance, resistance to environmental stress (e.g. nitrogen limited conditions), nitrogen fixation, fungal disease resistance, virus resistance, insect control, insect resistance, disease resistance, pathogen resistance, nematode resistance, bacterial disease resistance, starch production or content, oil production or content, processing qualities, fatty acid production or content, protein production or content, pharmaceutical peptides, flavor, fruit ripening, animal and human nutrition, seed production, fiber production, biopolymer production, or biofuel production.

Alternatively, a gene of agronomic interest can affect the above mentioned plant characteristic or phenotype by encoding an RNA molecule that causes the targeted modulation of gene expression of an endogenous gene, for example via antisense (see e.g. U.S. Pat. No. 5,107,065); inhibitory RNA (“RNAi”, including modulation of gene expression via miRNA-, siRNA-, trans-acting siRNA-, and phased sRNA-mediated mechanisms, e.g. as described in published applications US 2006/0200878 and US 2008/0066206, and in U.S. patent application Ser. No. 11/974,469); or co-suppression-mediated mechanisms. The RNA could also be a catalytic RNA molecule (e.g. a ribozyme or a riboswitch; see e.g. US 2006/0200878) engineered to cleave a desired endogenous mRNA product. Thus, any transcribable polynucleotide molecule that encodes a transcribed RNA molecule that affects an agronomically important phenotype or morphology change of interest may be useful for the practice of the present invention. Methods are known in the art for constructing and introducing constructs into a cell in such a manner that the transcribable polynucleotide molecule is transcribed into a molecule that is capable of causing gene suppression. For example, posttranscriptional gene suppression using a construct with an anti-sense oriented transcribable polynucleotide molecule to regulate gene expression in plant cells is disclosed in U.S. Pat. Nos. 5,107,065 and 5,759,829, and posttranscriptional gene suppression using a construct with a sense-oriented transcribable polynucleotide molecule to regulate gene expression in plants is disclosed in U.S. Pat. Nos. 5,283,184 and 5,231,020. Expression of a transcribable polynucleotide in a plant cell can also be used to suppress plant pests feeding on the plant cell, for example, compositions isolated from coleopteran pests (U.S. Patent Publication No. US20070124836) and compositions isolated from nematode pests (U.S. Patent Publication No. US20070250947). Plant pests include, but are not limited to arthropod pests, nematode pests, and fungal or microbial pests. Exemplary transcribable polynucleotide molecules for incorporation into constructs of the present invention include, for example, DNA molecules or genes from a species other than the target species or genes that originate with or are present in the same species, but are incorporated into recipient cells by genetic engineering methods rather than classical reproduction or breeding techniques. The type of polynucleotide molecule can include, but is not limited to, a polynucleotide molecule that is already present in the plant cell, a polynucleotide molecule from another plant, a polynucleotide molecule from a different organism, or a polynucleotide molecule generated externally, such as a polynucleotide molecule containing an antisense message of a gene, or a polynucleotide molecule encoding an artificial, synthetic, or otherwise modified version of a transgene.

Genes of interest can include, for example, those genes involved in information, such as zinc fingers, those involved in communication, such as kinases, and those involved in housekeeping, such as heat shock proteins; those involved in oil, starch, carbohydrate, or nutrient metabolism; genes encoding enzymes and other proteins from plants and other sources including prokaryotes and other eukaryotes.

Examples of genes of agronomic interest known in the art include those for herbicide resistance (U.S. Pat. Nos. 6,803,501; 6,448,476; 6,248,876; 6,225,114; 6,107,549; 5,866,775; 5,804,425; 5,633,435; and 5,463,175), increased yield (U.S. Pat. Nos. RE38,446; 6,716,474; 6,663,906; 6,476,295; 6,441,277; 6,423,828; 6,399,330; 6,372,211; 6,235,971; 6,222,098; and 5,716,837), insect control (U.S. Pat. Nos. 6,809,078; 6,713,063; 6,686,452; 6,657,046; 6,645,497; 6,642,030; 6,639,054; 6,620,988; 6,593,293; 6,555,655; 6,538,109; 6,537,756; 6,521,442; 6,501,009; 6,468,523; 6,326,351; 6,313,378; 6,284,949; 6,281,016; 6,248,536; 6,242,241; 6,221,649; 6,177,615; 6,156,573; 6,153,814; 6,110,464; 6,093,695; 6,063,756; 6,063,597; 6,023,013; 5,959,091; 5,942,664; 5,942,658, 5,880,275; 5,763,245; and 5,763,241), fungal disease resistance (U.S. Pat. Nos. 6,653,280; 6,573,361; 6,506,962; 6,316,407; 6,215,048; 5,516,671; 5,773,696; 6,121,436; 6,316,407; and 6,506,962), virus resistance (U.S. Pat. Nos. 6,617,496; 6,608,241; 6,015,940; 6,013,864; 5,850,023; and 5,304,730), nematode resistance (U.S. Pat. No. 6,228,992), bacterial disease resistance (U.S. Pat. No. 5,516,671), plant growth and development (U.S. Pat. Nos. 6,723,897 and 6,518,488), starch production (U.S. Pat. Nos. 6,538,181; 6,538,179; 6,538,178; 5,750,876; 6,476,295), modified oils production (U.S. Pat. Nos. 6,444,876; 6,426,447; and 6,380,462), high oil production (U.S. Pat. Nos. 6,495,739; 5,608,149; 6,483,008; and 6,476,295), modified fatty acid content (U.S. Pat. Nos. 6,828,475; 6,822,141; 6,770,465; 6,706,950; 6,660,849; 6,596,538; 6,589,767; 6,537,750; 6,489,461; and 6,459,018), high protein production (U.S. Pat. No. 6,380,466), fruit ripening (U.S. Pat. No. 5,512,466), enhanced animal and human nutrition (U.S. Pat. Nos. 6,723,837; 6,653,530; 6,541,259; 5,985,605; and 6,171,640), biopolymers (U.S. Pat. Nos. RE37,543; 6,228,623; and U.S. Pat. Nos. 5,958,745, and 6,946,588), environmental stress resistance (U.S. Pat. No. 6,072,103), pharmaceutical peptides and secretable peptides (U.S. Pat. Nos. 6,812,379; 6,774,283; 6,140,075; and 6,080,560), improved processing traits (U.S. Pat. No. 6,476,295), improved digestibility (U.S. Pat. No. 6,531,648) low raffinose (U.S. Pat. No. 6,166,292), industrial enzyme production (U.S. Pat. No. 5,543,576), improved flavor (U.S. Pat. No. 6,011,199), nitrogen fixation (U.S. Pat. No. 5,229,114), hybrid seed production (U.S. Pat. No. 5,689,041), fiber production (U.S. Pat. Nos. 6,576,818; 6,271,443; 5,981,834; and 5,869,720) and biofuel production (U.S. Pat. No. 5,998,700).

Transcribable polynucleotide molecules may also be markers useful in detecting transformed plant cells, plant tissue, plant parts, or plants described herein. As used herein the term “marker” refers to any transcribable polynucleotide molecule whose expression, or lack thereof, can be screened for or scored in some way. Marker genes for use in the practice of the present invention include, but are not limited to, transcribable polynucleotide molecules encoding β-glucuronidase (GUS described in U.S. Pat. No. 5,599,670), red fluorescent protein (e.g., mCherry), green fluorescent protein and variants and derivatives thereof (GFP described in U.S. Pat. Nos. 5,491,084 and 6,146,826), proteins that confer antibiotic resistance, or proteins that confer herbicide tolerance. Useful antibiotic resistance markers, including those encoding proteins conferring resistance to Basta (bar), kanamycin (nptII), hygromycin B (aph IV), streptomycin or spectinomycin (aad, spec/strep) and gentamycin (aac3 and aacC4) are known in the art. Herbicides for which transgenic plant tolerance has been demonstrated and the method of the present invention can be applied, include, but are not limited to, amino-methyl-phosphonic acid, glyphosate, glufosinate, sulfonylureas, imidazolinones, bromoxynil, dalapon, dicamba, cyclohexanedione, protoporphyrinogen oxidase inhibitors, and isoxasflutole herbicides.

Included within the term “selectable markers” are also genes which encode a secretable marker whose secretion can be detected as a means of identifying or selecting for transformed cells. Examples include markers that encode a secretable antigen that can be identified by antibody interaction, or even secretable enzymes which can be detected catalytically. Selectable secreted marker proteins fall into a number of classes, including small, diffusible proteins which are detectable, (e.g. by ELISA), small active enzymes which are detectable in extracellular solution (e.g, alpha-amylase, beta-lactamase, phosphinothricin transferase), or proteins which are inserted or trapped in the cell wall (such as proteins which include a leader sequence such as that found in the expression unit of extension or tobacco pathogenesis related proteins also known as tobacco PR-S).

“Selectable markers” can be used to distinguish between transformed and non-transformed genes. Reporter genes are test sequences whose expression can be quantified. Reporter genes can act as markers for transformed genes. In some embodiments, the transgenes of the present invention comprise at least one reporter gene. As used herein a “reporter” or a “reporter gene” refers to a nucleic acid molecule encoding a detectable marker. The reporter gene can be, for example, luciferase (e.g., firefly luciferase or Renilla luciferase), β-galactosidase, chloramphenicol acetyl transferase (CAT), or a fluorescent protein (e.g., green fluorescent protein (GFP), red fluorescent protein (DsRed), yellow fluorescent protein, blue fluorescent protein, cyan fluorescent protein, or variants thereof, including enhanced variants such as enhanced GFP (eGFP). Reporter genes are detectable by a reporter assay. Reporter assays can measure the level of reporter gene expression or activity by any number of means, including, for example, measuring the level of reporter mRNA, the level of reporter protein, or the amount of reporter protein activity. Reporter assays are known in the art or otherwise disclosed herein.

Reporter genes are test sequences whose expression can be quantified. Reporter genes can act as markers for transformed genes. In some embodiments, the transgenes of the present invention comprise at least one reporter gene. As used herein a “reporter” or a “reporter gene” refers to a nucleic acid molecule encoding a detectable marker. The reporter gene can be, for example, luciferase (e.g., firefly luciferase or Renilla luciferase), β-galactosidase, chloramphenicol acetyl transferase (CAT), or a fluorescent protein (e.g., green fluorescent protein (GFP), red fluorescent protein (DsRed), yellow fluorescent protein, blue fluorescent protein, cyan fluorescent protein, or variants thereof, including enhanced variants such as enhanced GFP (eGFP). Reporter genes are detectable by a reporter assay. Reporter assays can measure the level of reporter gene expression or activity by any number of means, including, for example, measuring the level of reporter mRNA, the level of reporter protein, or the amount of reporter protein activity. Reporter assays are known in the art or otherwise disclosed herein. Other possible selectable marker genes will be apparent to those of skill in the art and are encompassed by the present disclosure.

### Constructs

As used herein, the term “construct” means any recombinant polynucleotide molecule such as a plasmid, cosmid, virus, autonomously replicating polynucleotide molecule, phage, or linear or circular single-stranded or double-stranded DNA or RNA polynucleotide molecule, derived from any source, capable of genomic integration or autonomous replication, comprising a polynucleotide molecule where one or more polynucleotide molecule has been linked in a functionally operative manner, i.e. operably linked. As used herein, the term “vector” means any recombinant polynucleotide construct that may be used for the purpose of transformation, i.e., the introduction of heterologous DNA into a host cell. The term includes an expression cassette isolated from any of the aforementioned molecules.

As used herein, the term “operably linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operably linked to a transcribable polynucleotide molecule if the promoter modulates transcription of the transcribable polynucleotide molecule of interest in a cell. A leader, for example, is operably linked to coding sequence when it is capable of serving as a leader for the polypeptide encoded by the coding sequence.

The constructs of the present invention may be provided, in one embodiment, as double Ti plasmid border DNA constructs that have the right border (RB or AGRtu.RB) and left border (LB or AGRtu.LB) regions of the Ti plasmid isolated from Agrobacterium tumefaciens comprising a T-DNA, that along with transfer molecules provided by the A. tumefaciens cells, permit the integration of the T-DNA into the genome of a plant cell (see, for example, U.S. Pat. No. 6,603,061). The constructs may also contain the plasmid backbone DNA segments that provide replication function and antibiotic selection in bacterial cells, for example, an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. For plant transformation, the host bacterial strain is often A. tumefaciens ABI, C58, or LBA4404; however, other strains known to those skilled in the art of plant transformation can function in the present invention. For example, Agrobacterium rhizogenes ARqual.

Methods are known in the art for assembling and introducing constructs into a cell in such a manner that the transcribable polynucleotide molecule is transcribed into a functional mRNA molecule that is translated and expressed as a protein product. For the practice of the present invention, conventional compositions and methods for preparing and using constructs and host cells are well known to one skilled in the art, see, for example, Molecular Cloning: A Laboratory Manual, 3^{rd} edition Volumes 1, 2, and 3 (2000) J. Sambrook, D. W. Russell, and N. Irwin, Cold Spring Harbor Laboratory Press. Methods for making recombinant vectors particularly suited to plant transformation include, without limitation, those described in U.S. Pat. Nos. 4,971,908; 4,940,835; 4,769,061; and 4,757,011 in their entirety. These types of vectors have also been reviewed in the scientific literature (see, for example, Rodriguez, et al., Vectors: A Survey of Molecular Cloning Vectors and Their Uses, Butterworths, Boston, (1988) and Glick, et al., Methods in Plant Molecular Biology and Biotechnology, CRC Press, Boca Raton, FL. (1993)). Typical vectors useful for expression of nucleic acids in higher plants are well known in the art and include vectors derived from the tumor-inducing (Ti) plasmid of Agrobacterium tumefaciens (Rogers, et al., Methods in Enzymology 153: 253-277 (1987)). Other recombinant vectors useful for plant transformation, including the pCaMVCN transfer control vector, have also been described in the scientific literature (see, for example, Fromm, et al., Proc. Natl. Acad. Sci. USA 82: 5824-5828 (1985)).

Various regulatory elements may be included in a construct including any of those provided herein such as SEQ ID NO:4, or variants or fragments thereof. Any such regulatory elements may be provided in combination with other regulatory elements. Such combinations can be designed or modified to produce desirable regulatory features. In one embodiment, constructs of the present invention comprise at least one regulatory element operably linked to a transcribable polynucleotide molecule operably linked to a 3′ UTR.

As used herein, the term “3′ transcription termination molecule” or “3′ UTR” refers to a DNA molecule that is used during transcription to produce the 3′ untranslated region (3′ UTR) of an mRNA molecule. The 3′ untranslated region of an mRNA molecule may be generated by specific cleavage and 3′ polyadenylation, a.k.a. polyA tail. A 3′ UTR may be operably linked to and located downstream of a transcribable polynucleotide molecule and may include polynucleotides that provide a polyadenylation signal and other regulatory signals capable of affecting transcription, mRNA processing, or gene expression. PolyA tails are thought to function in mRNA stability and in initiation of translation. Examples of 3′ transcription termination molecules in the art are the nopaline synthase 3′ region (see, Fraley, et al., Proc. Natl. Acad. Sci. USA, 80: 4803-4807 (1983)); wheat hsp17 3′ region; pea rubisco small subunit 3′ region; cotton E6 3′ region (U.S. Pat. No. 6,096,950); 3′ regions disclosed in WO0011200A2; and the coixin 3′ UTR (U.S. Pat. No. 6,635,806).

Constructs of the present invention may include any promoter or fragment or variant thereof provided herein, such as SEQ ID NO:4, or known in the art. For example, a promoter of the present invention may be operably linked to a transcribable polynucleotide sequence, such as a sequence encoding one or more polypeptides selected from SEQ ID NOs: 5, 7, and 9, or variants or fragments thereof. Alternatively, a heterologous promoter such as the Cauliflower Mosaic Virus 35S transcript promoter (see, U.S. Pat. No. 5,352,605) or a heterologous promoter derived from a heat shock protein gene (see, U.S. Pat. Nos. 5,659,122 and 5,362,865) may be operably linked to a polypeptide sequence as disclosed herein.

A construct provided herein may further comprise additional elements useful in regulating or modulating expression of a transcribable polynucleotide, including leader, enhancer, intron, and 3′ UTR sequences. A construct provided herein may further comprise one or more marker sequences for identification of the construct in plant cells, plant tissue, or plants.

### Expression Cassette.

The polynucleotide of the present invention can be provided in expression cassettes for expression of a gene of interest in the plant or other organism or host cell of interest. It is recognized that the polynucleotide of the present invention and expression cassettes comprising them can be used for the expression in both human and non-human host cells including, but not limited to, host cells from plants, animals, fungi, and algae. In one embodiment of the invention, the host cells are human host cells or a host cell line that is incapable of differentiating into a human being.

The expression cassette can include 5′ and 3′ regulatory sequences operably linked to the gene of interest to be expressed. “Operably linked” is intended to mean a functional linkage between two or more elements. For example, an operable linkage between one or more genetic regulatory elements and a gene of interest is a functional link between the gene of interest and the one or more genetic regulatory elements that allows for expression of the gene of interest. Operably linked elements may be contiguous or non-contiguous. The cassette may additionally contain at least one additional gene to be cotransformed into the organism. Alternatively, the additional gene(s) can be provided on multiple expression cassettes. Such an expression cassette is provided with a plurality of restriction sites and/or recombination sites for insertion of the polynucleotide to be under the transcriptional regulation of the regulatory regions. The expression cassette may additionally contain selectable marker genes. The expression cassette can include, in the 5′-3′ direction of transcription, a transcriptional and translational initiation region (i.e., a promoter), polynucleotide to be expressed, and a transcriptional and translational termination region (i.e., termination region) functional in plants or other organism or host cell. The regulatory regions (i.e., promoters, transcriptional regulatory regions, and translational termination regions) and/or the polynucleotide to be expressed may be native/analogous to the host cell or to each other. The promoter may be provided by the polynucleotide of the invention in some embodiments.

Where appropriate, the genes of interest may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) PNAS USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The expression cassette can also comprise a selectable marker gene for the selection of transformed cells. Selectable marker genes are utilized for the selection of transformed cells or tissues. A selectable marker gene can be positively or negatively selectable. For positive selection, a foreign gene is supplied to a plant cell that allows it to utilize a substrate present in the medium that it otherwise could not use, such as mannose or xylose (for example, refer U.S. Pat. Nos. 5,767,378; 5,994,629). More typically, however, negative selection is used because it is more efficient, utilizing selective agents such as herbicides or antibiotics that either kill or inhibit the growth of nontransformed plant cells and reducing the possibility of chimeras. Non-limiting exemplary marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, sulfonylurea, glyphosate, glufosinate, L-phosphinothricin, triazine, benzonitrile and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng. 85:610-9 and Fetter et al. (2004) Plant Cell 16:215-28), cyan florescent protein (CYP) (Bolte et al. (2004) J. Cell Science 117:943-54 and Kato et al. (2002) Plant Physiol. 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) PNAS 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) PNAS 86:5400-5404; Fuerst et al. (1989) PNAS 86:2549-2553; Deuschle et al. (1990) Science 248:480-483; Gossen (1993) Ph.D. Thesis, University of Heidelberg; Reines et al. (1993) PNAS 90:1917-1921; Labow et al. (1990) Mol. Cell. Biol. 10:3343-3356; Zambretti et al. (1992) PNAS 89:3952-3956; Baim et al. (1991) PNAS 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) PNAS 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724; Bourouis et al., EMBO J. 2(7): 1099-1104 (1983) White et al., Nucl Acids Res 18: 1062 (1990), Spencer et al., Theon Appl Genet 79: 625-631(1990), U.S. Pat. Nos. 5,034,322; 6,174,724; 6,255,560; 4,795,855; 5,378,824; and 6,107,549. Such disclosures are herein incorporated by reference.

The above list of selectable marker genes is not limiting. Any selectable marker gene can be used in the present invention. Numerous plant transformation vectors and methods for transforming plants are available. See, for example, An, G. et al. (1986) Plant Pysiol., 81:301-305; Fry, J., et al. (1987) Plant Cell Rep. 6:321-325; Block, M. (1988) Theor. Appl Genet. 76:767-774; Hinchee, et al. (1990) Stadler. Genet. Symp. 203212.203-212; Cousins, et al. (1991) Aust. J. Plant Physiol. 18:481-494; Chee, P. P. and Slightom, J. L. (1992) Gene 118:255-260; Christou, et al. (1992) Trends. Biotechnol. 10:239-246; D'Halluin, et al. (1992) Bio/Technol. 10:309-314; Dhir, et al. (1992) Plant Physiol. 99:81-88; Casas et al. (1993) PNAS 90:11212-11216; Christou, P. (1993) In Vitro Cell. Dev. Biol.-Plant; 29P:119-124; Davies, et al. (1993) Plant Cell Rep. 12:180-183; Dong, J. A. and Mchughen, A. (1993) Plant Sci. 91:139-148; Franklin, C. I. and Trieu, T. N. (1993) Plant. Physiol. 102:167; Golovkin, et al. (1993) Plant Sci. 90:41-52; Guo Chin Sci. Bull. 38:2072-2078; Asano, et al. (1994) Plant Cell Rep. 13; Ayeres N. M. and Park, W. D. (1994) Crit. Rev. Plant. Sci. 13:219-239; Barcelo, et al. (1994) Plant. J. 5:583-592; Becker, et al. (1994) Plant. J. 5:299-307; Borkowska et al. (1994) Acta. Physiol Plant. 16:225-230; Christou, P. (1994) Agro. Food. Ind. Hi Tech. 5: 17-27; Eapen et al. (1994) Plant Cell Rep. 13:582-586; Hartman, et al. (1994) Bio-Technology 12: 919923; Ritala, et al. (1994) Plant. Mol. Biol. 24:317-325; and Wan, Y. C. and Lemaux, P. G. (1994) Plant Physiol. 104:3748.

For the transformation of plants and plant cells, the nucleotide sequences of the invention are inserted using standard techniques into any vector known in the art that is suitable for expression of the nucleotide sequences in a plant or plant cell. The selection of the vector depends on the preferred transformation technique and the target plant species to be transformed.

Methodologies for constructing plant expression cassettes and introducing foreign nucleic acids into plants are generally known in the art and have been previously described. For example, foreign DNA can be introduced into plants, using tumor-inducing (Ti) plasmid vectors. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. Nos. 5,693,512, 6,051,757 and EP904362A1. Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Methods of Agrobacterium-mediated plant transformation that involve using vectors with no T-DNA are also well known to those skilled in the art and can have applicability in the present invention. See, for example, U.S. Pat. No. 7,250,554, which utilizes P-DNA instead of T-DNA in the transformation vector. Agrobacterium tumefaciens is a naturally occurring bacterium that is capable of inserting its DNA (genetic information) into plants, resulting in a type of injury to the plant known as crown gall. A transgenic plant formed using Agrobacterium transformation methods typically contains a single gene on one chromosome, although multiple copies are possible. Such transgenic plants can be referred to as being hemizygous for the added gene.

Other methods utilized for the delivery foreign DNA or other foreign nucleic acids involve the use of PEG mediated protoplast transformation, electroporation, microinjection whiskers, and biolistics or microprojectile bombardment for direct DNA uptake. Such methods are known in the art. (U.S. Pat. No. 5,405,765 to Vasil et al.; Bilang et al. (1991) Gene 100: 247-250; Scheid et al., (1991) Mol. Gen. Genet. 228: 104-112; Guerche et al., (1987) Plant Science 52: 111-116; Neuhaus et al., (1987) Theor. Appl Genet. 75: 30-36; Klein et al., (1987) Nature 327: 70-73; Howell et al., (1980) Science 208:1265; Horsch et al., (1985) Science 227: 1229-1231; DeBlock et al., (1989) Plant Physiology 91: 694-701; Methods for Plant Molecular Biology (Weissbach and Weissbach, eds.) Academic Press, Inc. (1988); Methods in Plant Molecular Biology (Schuler and Zielinski, eds.) Academic Press, Inc. (1989); M. E. Fromm et al., Nature, 319, 791 (1986); H. Jones et al., Plant Mol. Biol., 13, 501 (1989) and H. Yang et al., Plant Cell Reports, 7, 421 (1988); UMizuno et al., 2004; Petolino et al., 2000; U.S. Pat. No. 5,302,523; and US Application Publication No. 20040197909; Kaepler et al., 1992; Raloff, 1990; Wang, 1995; U.S. Pat. Nos. 5,204,253; 5,015,580; 5,405,765; 5,472,869; 5,538,877; 5,538,880; 5,550,318; 5,641,664; 5,736,369 and 5,736,369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and Raineri et al., Bio/Tech. 8:33-38 (1990), each of which is incorporated herein by reference in its entirety). The method of transformation depends upon the plant cell to be transformed, stability of vectors used, expression level of gene products and other parameters. Specific methods for transforming certain plant species (e.g., maize, rice, wheat, barley, soybean) are described in U.S. Pat. Nos. 4,940,838, 5,464,763, 5,149,645, 5,501,967, 6,265,638, 4,693,976, 5,635,381, 5,731,179, 5,693,512, 6,162,965, 5,693,512, 5,981,840, 6,420,630, 6,919,494, 6,329,571, 6,215,051, 6,369,298, 5,169,770, 5,376,543, 5,416,011, 5,569,834, 5,824,877, 5,959,179, 5,563,055, and 5,968,830, each of which is incorporated by reference in its entirety.

Other suitable methods of introducing nucleotide sequences into plant cells and subsequent insertion into the plant genome include microinjection as Crossway et al. (1986) Biotechniques 4:320-334, electroporation as described by Riggs et al. (1986) PNAS 83:5602-5606, Agrobacterium-mediated transformation as described by Townsend et al., U.S. Pat. No. 5,563,055, Zhao et al., U.S. Pat. No. 5,981,840, Yukon et al., WO 94/000977, and Hideaki et al., WO 95/06722, direct gene transfer as described by Paszkowski et al. (1984) EMBO J. 3:2717-2722, and ballistic particle acceleration as described in, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec transformation (WO 00/28058). Also see, Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) PNAS 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) PNAS 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The polynucleotides of the invention may be introduced into plants by contacting plants with a virus or viral nucleic acids. Generally, such methods involve incorporating a polynucleotide construct of the invention within a viral DNA or RNA molecule. Further, it is recognized that promoters of the invention also encompass promoters utilized for transcription by viral RNA polymerases. Methods for introducing polynucleotide constructs into plants and expressing a protein encoded therein, involving viral DNA or RNA molecules, are known in the art. See, for example, U.S. Pat. Nos. 5,889,191, 5,889,190, 5,866,785, 5,589,367 and 5,316,931; herein incorporated by reference.

In some embodiments, the polynucleotides of the invention may be introduced into plants using a sexual cross between two lines, and then repeated back-crossing between hybrid offspring and one of the parents until a plant with the desired characteristics is obtained. This process, however, is restricted to plants that can sexually hybridize, and genes in addition to the desired gene will be transferred.

Recombinant DNA techniques allow plant researchers to circumvent these limitations by enabling plant geneticists to identify and clone specific genes for desirable traits, such as resistance to an insect pest, and to introduce these genes into already useful varieties of plants. Once the foreign genes have been introduced into a plant, that plant can then be used in conventional plant breeding schemes (e.g., pedigree breeding, single-seed-descent breeding schemes, reciprocal recurrent selection) to produce progeny which also contain the gene of interest.

In some embodiments, genes can be introduced in a site directed fashion using homologous recombination. Homologous recombination permits site specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

In this manner, the present invention provides transformed seed (also referred to as “transgenic seed”) having a polynucleotide construct of the invention, for example, an expression cassette of the invention, stably incorporated into their genome.

In specific embodiments, the nucleic acid molecules and polynucleotide constructs of the present invention can be provided to a plant using a variety of transient transformation methods. Such transient transformation methods include, but are not limited to, the introduction of the sequence or variants and fragments thereof directly into the plant or the introduction of a transcript into the plant. Such methods include, for example, microinjection, electroporation, or particle bombardment. See, for example, Crossway et al. (1986) Mol Gen. Genet. 202:179-185; Nomura et al. (1986) Plant Sci. 44:53-58; Hepler et al. (1994) PNAS 91: 2176-2180 and Hush et al. (1994) The Journal of Cell Science 107:775-784, Sheen, J. 2002. A transient expression assay using maize mesophyll protoplasts. Sheen, J. 2001. Signal transduction in maize and Arabidopsis mesophyll protoplasts. Plant Physiol. 2001 December; 127:1466-1475, Anderson et al., U.S. Pat. No. 7,645,919 B2, all of which are herein incorporated by reference. Alternatively, the polynucleotide can be transiently transformed into the plant using techniques known in the art.

### Regulatory Elements

Regulatory elements are genetic elements that regulate gene activity by modulating the transcription of an operably linked transcribable polynucleotide molecule. Such elements include promoters, leaders, introns, and 3′ untranslated regions and are useful in the field of plant molecular biology and plant genetic engineering.

A regulatory element is a DNA molecule having gene regulatory activity, i.e., one that has the ability to affect the transcription and/or translation of an operably linked transcribable polynucleotide molecule. The term “gene regulatory activity” thus refers to the ability to affect the expression pattern of an operably linked transcribable polynucleotide molecule by affecting the transcription and/or translation of that operably linked transcribable polynucleotide molecule. As used herein, a regulatory element may be comprised of expression elements, such as enhancers, promoters, and introns, operably linked. A regulatory element may also be comprised of leaders, 3′ untranslated regions (3′ UTRs), terminators, transcription termination regions, or chromatin control. Regulatory elements, capable of providing a unique spatial and temporal expression profile to an operably linked heterologous transcribable polynucleotide molecule are therefore useful for modifying plant phenotypes through the methods of genetic engineering. Thus, a transcriptional regulatory sequence may be comprised of, for instance, a promoter operably linked 5′ to a leader sequence, which is in turn operably linked 5′ to an intron sequence. Leaders and introns can positively affect the transcription of an operably linked transcribable polynucleotide molecule as well as the translation of the resulting transcribed RNA. The pre-processed RNA molecule comprising leaders and introns, can affect the post-transcriptional processing of the transcribed RNA and/or the export of the transcribed RNA molecule from the cell nucleus into the cytoplasm. Following post-transcriptional processing of the transcribed RNA molecule, the leader sequence may be retained as part of the final messenger RNA and may positively affect the translation of the messenger RNA molecule. Regulatory elements include SEQ ID NO: 4 provided herein, or variants and fragments thereof.

Regulatory elements such as promoters, enhancers, leaders, such as 5′-untranslated regions or part thereof, introns, 3′-untranslated regions or part thereof, transcription termination regions (or 3′ UTRs), or chromatin control elements are DNA molecules that have gene regulatory activity and play an integral part in the overall expression of genes in living cells. The term “regulatory element” refers to a DNA molecule having gene regulatory activity, i.e. one that has the ability to affect the transcription and/or translation of an operably linked transcribable polynucleotide molecule. Isolated regulatory elements, such as promoters and leaders that function in plants are therefore useful for modifying plant phenotypes through the methods of genetic engineering.

It is recognized that polynucleotides of the present invention can comprise a plurality of regulatory elements such as, for example, a promoter and an enhancer. It is further recognized that some genetic regulatory elements act in concert with other genetic regulatory elements to control the regulation of an operably linked gene of interest. Moreover, it is recognized that some genetic regulatory elements such as, for example, a promoter or enhancer, can be separated from the transcribed region of a gene of interest by 1, 2, 3, or more kilobases of DNA.

The present invention also provides methods for controlling gene expression. “Controlling gene expression” refers to controlling the expression of an RNA transcript, and can further encompass translation of the transcript, or even an activity or function of the encoded protein. Controlling gene expression can include affecting one or more of RNA transcription, processing, turnover, and/or translation.

The genetic regulatory elements as disclosed herein can be implemented as regulatory sequences to control gene expression in a “desired manner.” The desired manner of gene expression can be temporally, spatially, or any combination thereof in a target organism including, but not limited to, constitutive expression, tissue-preferred expression, and organ-preferred expression. The desired manner of gene expression can also be expression in response to biotic stress (e.g., fungal, bacterial, and viral pathogens, insects, herbivores, and the like) and/or abiotic stress (e.g., wounding, drought, cold, heat, high nutrient levels, low nutrient levels, metals, light, herbicides and other synthetic chemicals, and the like). Regulatory elements may be characterized by their expression pattern effects (qualitatively and/or quantitatively), e.g., positive or negative effects and/or constitutive or other effects such as by their temporal, spatial, developmental, tissue, environmental, physiological, pathological, cell cycle, and/or chemically responsive expression pattern, and any combination thereof, as well as by quantitative or qualitative indications. A promoter is useful as a regulatory element for modulating the expression of an operably linked transcribable polynucleotide molecule.

As used herein, a “gene expression pattern” is any pattern of transcription of an operably linked DNA molecule into a transcribed RNA molecule. The transcribed RNA molecule may be translated to produce a protein molecule or may provide an antisense or other regulatory RNA molecule, such as a dsRNA, a tRNA, an rRNA, a miRNA, and the like.

As used herein, the term “expression pattern” or “expression profile” is any pattern of translation of a transcribed RNA molecule into a protein molecule. Protein expression may be characterized by its temporal, spatial, developmental, or morphological qualities as well as by quantitative or qualitative indications.

The transcribable DNA molecule may generally be any DNA molecule for which expression of a transcript is desired. Such expression of a transcript may result in translation of the resulting mRNA molecule, and thus protein expression. Alternatively, for example, a transcribable DNA molecule may be designed to ultimately cause decreased expression of a specific gene or protein. In one embodiment, this may be accomplished by using a transcribable DNA molecule that is oriented in the antisense direction. One of ordinary skill in the art is familiar with using such antisense technology. Any gene may be negatively regulated in this manner, and, in one embodiment, a transcribable DNA molecule may be designed for suppression of a specific gene through expression of a dsRNA, siRNA, or miRNA molecule.

As used herein, the term “promoter” refers generally to a DNA molecule that is involved in recognition and binding of RNA polymerase II and other proteins (trans-acting transcription factors) to initiate transcription. A promoter may be initially isolated from the 5′ untranslated region (5′ UTR) of a genomic copy of a gene. Alternately, promoters may be synthetically produced or manipulated DNA molecules. Promoters may also be chimeric, that is a promoter produced through the fusion of two or more heterologous DNA molecules. Promoters useful in practicing the present invention include SEQ ID NO:4, or variants or fragments thereof.

In specific embodiments of the invention, such molecules and any variants or derivatives thereof as described herein, are further defined as comprising promoter activity, i.e., are capable of acting as a promoter in a host cell, such as in a transgenic plant. In still further specific embodiments, a fragment may be defined as exhibiting promoter activity possessed by the starting promoter molecule from which it is derived, or a fragment may comprise a “minimal promoter” which provides a basal level of transcription and is comprised of a TATA box or equivalent sequence for recognition and binding of the RNA polymerase II complex for initiation of transcription.

In accordance with the invention a promoter or promoter fragment may be analyzed for the presence of known promoter elements, i.e., DNA sequence characteristics, such as a TATA-box and other known transcription factor binding site motifs. Identification of such known promoter elements may be used by one of skill in the art to design variants of the promoter having a similar expression pattern to the original promoter.

In some embodiments, the present disclosure provides polynucleotides containing promoters and/or enhancers. “Promoter” refers to a nucleotide sequence that is capable of controlling the expression of an operably linked coding sequence or other sequence encoding an RNA that is not necessarily translated into a protein. Thus, the polynucleotide may comprise proximal promoter elements as well as more distal upstream elements, the latter elements often referred to as enhancers. An “enhancer” refers to a DNA sequence that can stimulate promoter activity and may be an innate element of the promoter or a heterologous element inserted to enhance the level or tissue-specificity of a promoter. It is understood by those skilled in the art that different promoters may direct the expression of a gene in different tissues or cell types, at different stages of development, or in response to different environmental conditions. It is further recognized that because in most cases the exact boundaries of regulatory sequences have not been completely defined, nucleic acid fragments of some variation may have identical or similar promoter activity.

Promoters that cause a gene to be expressed in most cell types of an organism and at most times are commonly referred to as “constitutive promoters”. Expression of a gene in most cell types of an organism and at most times is referred to herein as “constitutive gene expression” or “constitutive expression”.

As used herein, the term “intron” refers to a DNA molecule that may be isolated or identified from the genomic copy of a gene and may be defined generally as a region spliced out during mRNA processing prior to translation. Alternately, an intron may be a synthetically produced or manipulated DNA element. An intron may contain enhancer elements that effect the transcription of operably linked genes. An intron may be used as a regulatory element for modulating expression of an operably linked transcribable polynucleotide molecule. A DNA construct may comprise an intron, and the intron may or may not be heterologous with respect to the transcribable polynucleotide molecule sequence. Examples of introns in the art include the rice actin intron (U.S. Pat. No. 5,641,876) and the corn HSP70 intron (U.S. Pat. No. 5,859,347). Further, when modifying intron/exon boundary sequences, it may be preferable to avoid using the nucleotide sequence AT or the nucleotide A just prior to the 5′ end of the splice site (GT) and the nucleotide G or the nucleotide sequence TG, respectively just after 3′ end of the splice site (AG) to eliminate the potential of unwanted start codons from being formed during processing of the messenger RNA into the final transcript. The sequence around the 5′ or 3′ end splice junction sites of the intron can thus be modified in this manner.

In some embodiments, the regulatory element is an expression-enhancing intron. An “expression-enhancing intron” or “enhancing intron” is an intron that is capable of causing an increase in the expression of a gene to which it is operably linked. While the present invention is not known to depend on a particular biological mechanism, it is believed that the expression-enhancing introns of the present invention enhance expression through intron mediated enhancement (IME). It is recognized that naturally occurring introns that enhance expression through IME are typically found within 1 Kb of the transcription start site of their native genes (see, Rose et al. (2008) Plant Cell 20:543-551). Such introns are usually the first intron, whether the first intron is in the 5′ UTR or the coding sequence, and need to be in a transcribed region. Introns that enhance expression solely through IME do not enhance gene expression when they are inserted into a non-transcribed region of a gene, such as, for example, a promoter. That is, they do not function as transcriptional enhancers. Unless stated otherwise or apparent from the context, the expression-enhancing introns of the present invention are capable of enhancing gene expression when they are found in a transcribed region of a gene but not when they occur in a non-transcribed region such as, for example, a promoter.

In some embodiments, the promoter is a plant promoter. A “plant promoter” is a promoter capable of initiating transcription in plant cells whether or not its origin is a plant cell, e.g., it is well known that Agrobacterium promoters are functional in plant cells. Thus, plant promoters include promoter DNA obtained from plants, plant viruses and bacteria such as Agrobacterium and Bradyrhizobium bacteria, and synthetic promoters capable of initiating transcription in plant cells. A plant promoter can be a constitutive promoter, a non-constitutive promoter, an inducible promoter, a repressible promoter, a tissue specific promoter (e.g., a root specific promoter, a stem specific promoter, a leaf specific promoter), a tissue preferred promoter (e.g., a root preferred promoter, a stem preferred promoter, a leaf preferred promoter), a cell type specific or preferred promoter (e.g., a meristem cell specific/preferred promoter), or many other types. In some embodiments, the variant polynucleotides or fragments described herein include additional known cis-acting sequences to drive expression of a transcribed gene in a desired manner.

In some embodiments, the promoter is a constitutive promoter. A “constitutive promoter” is a promoter which is active under most conditions and/or during most development stages. There are several advantages to using constitutive promoters in expression vectors used in plant biotechnology, such as: high level of production of proteins used to select transgenic cells or plants; high level of expression of reporter proteins or scorable markers, allowing easy detection and quantification; high level of production of a transcription factor that is part of a regulatory transcription system; production of compounds that requires ubiquitous activity in the plant; and production of compounds that are required during all stages of plant development. For illustration, constitutive promoters can include CaMV 19S promoter, CaMV 35S promoter (U.S. Pat. Nos. 5,352,605; 5,530,196 and 5,858,742), opine promoters, ubiquitin promoter, actin promoter, alcohol dehydrogenase promoter, etc. In some embodiments, the synthetic promoter prepared as described herein, is used to drive expression of a heterologous sequence, while CaMV 35S promoter is used to drive expression of a second sequence.

In some embodiments, the promoter is a non-constitutive promoter. A “non-constitutive promoter” is a promoter which is active under certain conditions, in certain types of cells, and/or during certain development stages. For example, tissue specific, tissue preferred, cell type specific, cell type preferred, inducible promoters, and promoters under developmental control are non-constitutive promoters. Examples of promoters under developmental control include promoters that preferentially initiate transcription in certain tissues, such as stems, leaves, roots, or seeds.

In some embodiments, the promoter is an inducible or a repressible promoter. An “inducible” or “repressible” promoter is a promoter which is under chemical or environmental factor control. Examples of environmental conditions that may affect transcription by inducible promoters include cold, heat, drought, certain chemicals, or the presence of light.

In some embodiments, the promoter is a tissue-specific promoter. A “tissue-specific” promoter is a promoter that initiates transcription only in certain tissues. Unlike constitutive expression of genes, tissue-specific expression is the result of several interacting levels of gene regulation. As such, in the art sometimes it is preferable to use promoters from homologous or closely related plant species to achieve efficient and reliable expression of transgenes in particular tissues. This is one of the main reasons for the large number of tissue-specific promoters isolated from particular plants and tissues found in both scientific and patent literature. Non-limiting examples of known tissue-specific promoters can include beta-amylase gene or barley hordein gene promoters (for seed gene expression), tomato pz7 and pz130 gene promoters (for ovary gene expression), tobacco RD2 gene promoter (for root gene expression), banana TRX promoter and melon actin promoter (for fruit gene expression), and embryo specific promoters, e.g., a promoter associated with an amino acid permease gene (AAP1), an oleate 12-hydroxylase:desaturase gene from Lesquerella fendleri (LFAH12), an 2S2 albumin gene (2S2), a fatty acid elongase gene (FAE1), or a leafy cotyledon gene (LEC2).

In some embodiments, the promoter is a tissue-preferred promoter. A “tissue-preferred” promoter is a promoter that initiates transcription mostly, but not necessarily entirely or solely in certain tissues.

In some embodiments, the promoter is a cell-type-specific promoter. A “cell-type-specific” promoter is a promoter that primarily drives expression in certain cell types in one or more organs, for example, vascular cells in roots, leaves, stalk cells, and stem cells.

In some embodiments, the promoter is a cell-type-preferred promoter. A “cell-type preferred” promoter is a promoter that primarily drives expression mostly, but not necessarily entirely or solely in certain cell types in one or more organs, for example, vascular cells in roots, leaves, stalk cells, and stem cells.

In some embodiments, the promoter is a root-specific promoter. A “root-specific” promoter is a promoter that initiates transcription only in root tissues.

In some embodiments, the promoter is a root-preferred promoter. A “root-preferred” promoter is a promoter that initiates transcription mostly, but not necessarily entirely or solely in root tissues.

In some embodiments, the present invention provides for methods to obtain inbred plants comprising the polynucleotide sequences. As used herein, the term “inbred” or “inbred plant” is used in the context of the present invention. This also includes any single gene conversions of that inbred. The phrase “single allele converted plant” as used herein refers to those plants which are developed by a plant breeding technique called backcrossing wherein essentially all of the desired morphological and physiological characteristics of an inbred are recovered in addition to the single allele transferred into the inbred via the backcrossing technique.

In one embodiment, fragments are provided of a promoter sequence disclosed herein. Promoter fragments may comprise promoter activity, as described above, and may be useful alone or in combination with other promoters and promoter fragments, such as in constructing chimeric promoters. In specific embodiments, fragments of a promoter are provided comprising at least about 50, 95, 150, 250, 500, 750, 1000, 1250, 1500, 1750, or at least about 2000 contiguous nucleotides, or longer, of a polynucleotide molecule having promoter activity disclosed herein.

Compositions derived from SEQ ID NO:4, such as internal or 5′ deletions, for example, can be produced using methods known in the art to improve or alter expression, including by removing elements that have either positive or negative effects on expression; duplicating elements that have positive or negative effects on expression; and/or duplicating or removing elements that have tissue or cell specific effects on expression. Compositions derived from SEQ ID NO:4 comprised of 3′ deletions in which the TATA box element or equivalent sequence thereof and downstream sequence is removed can be used, for example, to make enhancer elements. Further deletions can be made to remove any elements that have positive or negative; tissue specific; cell specific; or timing specific (such as, but not limited to, circadian rhythms) effects on expression. The promoter presented as SEQ ID NO:4 and fragments or enhancers derived therefrom can be used to make chimeric transcriptional regulatory element compositions comprised of SEQ ID NO:4 and the fragments or enhancers derived therefrom operably linked to other enhancers and promoters. The efficacy of the modifications, duplications, or deletions described herein on the desired expression aspects of a particular transgene may be tested empirically in stable and transient plant assays, such as those described in the working examples herein, so as to validate the results, which may vary depending upon the changes made and the goal of the change in the starting molecule.

As used herein, the term “leader” refers to a DNA molecule isolated from the untranslated 5′ region (5′ UTR) of a genomic copy of a gene and defined generally as a nucleotide segment between the transcription start site (TSS) and the protein coding sequence start site. Alternately, leaders may be synthetically produced or manipulated DNA elements. A leader can be used as a 5′ regulatory element for modulating expression of an operably linked transcribable polynucleotide molecule. Leader molecules may be used with a heterologous promoter or with their native promoter. Promoter molecules of the present invention may thus be operably linked to their native leader or may be operably linked to a heterologous leader. Leaders known in the art may be useful in practicing the present invention. The leader sequences (5′ UTR) may be comprised of regulatory elements or may adopt secondary structures that can have an effect on transcription or translation of a transgene. Leader sequences known in the art can be used in accordance with the invention to make chimeric regulatory elements that affect transcription or translation of a transgene. In addition, leader sequences can be used to make chimeric leader sequences that affect transcription or translation of a transgene.

The introduction of a foreign gene into a new plant host does not always result in a high expression of the incoming gene. Furthermore, if dealing with complex traits, it is sometimes necessary to modulate several genes with spatially or a temporarily different expression pattern. Introns can principally provide such modulation. However multiple uses of the same intron in one plant have shown to exhibit disadvantages. In those cases, it is necessary to have a collection of basic control elements for the construction of appropriate recombinant DNA elements.

As used herein, the term “enhancer” or “enhancer element” refers to a cis-acting transcriptional regulatory element, a.k.a. cis-element, which confers an aspect of the overall expression pattern, but is usually insufficient alone to drive transcription, of an operably linked polynucleotide sequence. Unlike promoters, enhancer elements do not usually include a transcription start site (TSS) or TATA box or equivalent sequence. A promoter may naturally comprise one or more enhancer elements that affect the transcription of an operably linked polynucleotide sequence. An isolated enhancer element may also be fused to a promoter to produce a chimeric promoter cis-element, which confers an aspect of the overall modulation of gene expression. A promoter or promoter fragment may comprise one or more enhancer elements that effect the transcription of operably linked genes. Many promoter enhancer elements are believed to bind DNA-binding proteins and/or affect DNA topology, producing local conformations that selectively allow or restrict access of RNA polymerase to the DNA template or that facilitate selective opening of the double helix at the site of transcriptional initiation. An enhancer element may function to bind transcription factors that regulate transcription. Some enhancer elements bind more than one transcription factor, and transcription factors may interact with different affinities with more than one enhancer domain. Enhancer elements can be identified by a number of techniques, including deletion analysis, i.e. deleting one or more nucleotides from the 5′ end or internal to a promoter; DNA binding protein analysis using DNase I footprinting, methylation interference, electrophoresis mobility-shift assays, in vivo genomic footprinting by ligation-mediated PCR, and other conventional assays; or by DNA sequence similarity analysis using known cis-element motifs or enhancer elements as a target sequence or target motif with conventional DNA sequence comparison methods, such as BLAST. The fine structure of an enhancer domain can be further studied by mutagenesis (or substitution) of one or more nucleotides or by other conventional methods. Enhancer elements can be obtained by chemical synthesis or by isolation from regulatory elements that include such elements, and they can be synthesized with additional flanking nucleotides that contain useful restriction enzyme sites to facilitate subsequence manipulation. Thus, the design, construction, and use of enhancer elements according to the methods disclosed herein for modulating the expression of operably linked transcribable polynucleotide molecules are encompassed by the present invention. Enhancer sequences derived from the CaMV can also be utilized (U.S. Pat. Nos. 5,164,316; 5,196,525; 5,322,938; 5,530,196; 5,352,605; 5,359,142; and 5,858,742 for example).

In plants, the inclusion of some introns in gene constructs leads to increased mRNA and protein accumulation relative to constructs lacking the intron.

This effect has been termed “intron mediated enhancement” (IME) of gene expression (Mascarenhas et al., (1990) Plant Mol. Biol. 15:913-920). Introns known to stimulate expression in plants have been identified in maize genes (e.g. tubA1, Adh1, Sh1, Ubi1 (Jeon et al. (2000) Plant Physiol. 123:1005-1014; Callis et al. (1987) Genes Dev. 1:1183-1200; Vasil et al. (1989) Plant Physiol. 91:1575-1579; Christiansen et al. (1992) Plant Mol. Biol. 18:675-689) and in rice genes (e.g. salt, tpi: McElroy et al., Plant Cell 2:163-171 (1990); Xu et al., Plant Physiol. 106:459-467 (1994)). Similarly, introns from dicotyledonous plant genes like those from petunia (e.g. rbcS), potato (e.g. st-ls1), and Arabidopsis thaliana (e.g. ubq3 and pat1) have been found to elevate gene expression rates (Dean et al. (1989) Plant Cell 1:201-208; Leon et al. (1991) Plant Physiol. 95:968-972; Norris et al. (1993) Plant Mol Biol 21:895-906; Rose and Last (1997) Plant J. 11:455-464). It has been shown that deletions or mutations within the splice sites of an intron reduce gene expression, indicating that splicing might be needed for IME (Mascarenhas et al. (1990) Plant Mol Biol. 15:913-920; Clancy and Hannah (2002) Plant Physiol. 130:918-929). However, that splicing per se is not required for a certain IME in dicotyledonous plants has been shown by point mutations within the splice sites of the pat1 gene from A. thaliana (Rose and Beliakoff (2000) Plant Physiol. 122:535-542).

Enhancement of gene expression by introns is not a general phenomenon because some intron insertions into recombinant expression cassettes fail to enhance expression (e.g. introns from dicot genes (rbcS gene from pea, phaseolin gene from bean and the stls-1 gene from Solanum tuberosum) and introns from maize genes (adh1 gene the ninth intron, hsp81 gene the first intron)) (Chee et al. (1986) Gene 41:47-57; Kuhlemeier et al. (1988) Mol Gen Genet 212:405-411; Mascarenhas et al. (1990) Plant Mol. Biol. 15:913-920; Sinibaldi and Mettler (1992) In WE Cohn, K Moldave, eds, Progress in Nucleic Acid Research and Molecular Biology, Vol 42. Academic Press, New York, pp 229-257; Vancanneyt et al. 1990 Mol. Gen. Genet. 220:245-250). Therefore, not each intron can be employed in order to manipulate the gene expression level of non-endogenous genes or endogenous genes in transgenic plants. What characteristics or specific sequence features must be present in an intron sequence in order to enhance the expression rate of a given gene is not known in the prior art and therefore from the prior art it is not possible to predict whether a given plant intron, when used heterologously, will cause enhancement of expression at the DNA level or at the transcript level (IME).

In one embodiment variants of the disclosed promoter sequences are provided. For example, a recombinant DNA molecule comprising a sequence that when optimally aligned to a reference sequence, such as SEQ ID NO:4, has at least about 85 percent identity, at least about 90 percent identity, at least about 95 percent identity, at least about 96 percent identity, at least about 97 percent identity, at least about 98 percent identity, or at least about 99 percent identity to the reference sequence. In particular embodiments such sequences may be defined as having the activity of the reference sequence, for example the activity of SEQ ID NO:4.

Also provided are fragments of regulatory sequences provided herein, for example fragments of a polynucleotide sequence of SEQ ID NO:4. In specific embodiments, fragments of a polynucleotide sequences are provided comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of SEQ ID NO:4 or another regulatory sequence disclosed herein. Methods for producing such fragments from a starting molecule are well known in the art. Fragments of a polynucleotide sequence provided herein may comprise the activity of the base sequence.

As used herein, the term “chimeric” refers to a single DNA molecule produced by fusing a first DNA molecule to a second DNA molecule, where neither the first nor second DNA molecule would normally be found in that configuration, i.e., fused to the other. The chimeric DNA molecule is thus a new DNA molecule not otherwise normally found in nature. As used herein, the term “chimeric promoter” refers to a promoter produced through such manipulation of DNA molecules. A chimeric promoter may combine two or more DNA fragments; an example would be the fusion of a promoter to an enhancer element. Thus, the design, construction, and use of chimeric promoters according to the methods disclosed herein for modulating the expression of operably linked transcribable polynucleotide molecules are encompassed by the present disclosure.

As used herein, the term “variant” refers to a second DNA molecule that is in composition similar, but not identical to, a first DNA molecule and yet the second DNA molecule still maintains the general functionality, i.e. same or similar expression pattern or encodes a polypeptide having similar activity to that of the first DNA molecule. A variant may be a shorter or truncated version of the first DNA molecule and/or an altered version of the sequence of the first DNA molecule, such as one with different restriction enzyme sites and/or internal deletions, substitutions, and/or insertions. A “variant” can also encompass a regulatory element having a nucleotide sequence comprising a substitution, deletion, and/or insertion of one or more nucleotides of a reference sequence, wherein the derivative regulatory element has more or less or equivalent transcriptional or translational activity than the corresponding parent regulatory molecule. The regulatory element “variants” will also encompass variants arising from mutations that naturally occur in bacterial and plant cell transformation. In the present invention, a polynucleotide sequence provided as SEQ ID NOs: 1-4, 6, or 8 may be used to create variants that are in composition similar, but not identical to, the polynucleotide sequence of the original regulatory element, while still maintaining the general functionality, i.e. same or similar expression pattern, of the original regulatory element. Production of such variants of the present invention is well within the ordinary skill of the art in light of the disclosure and is encompassed within the scope of the present invention. Chimeric regulatory element “variants” comprise the same constituent elements as a reference sequence but the constituent elements comprising the chimeric regulatory element may be operatively linked by various methods known in the art, such as, restriction enzyme digestion and ligation, ligation independent cloning, modular assembly of PCR products during amplification, or direct chemical synthesis of the regulatory element as well as other methods known in the art. The resulting chimeric regulatory element “variant” can be comprised of the same, or variants of the same, constituent elements of the reference sequence but differ in the sequence or sequences that comprise the linking sequence or sequences which allow the constituent parts to be operatively linked. In the present invention, a polynucleotide sequence provided as SEQ ID NOs: 1-4, 6, or 8 provides a reference sequence wherein the constituent elements that comprise the reference sequence may be joined by methods known in the art and may comprise substitutions, deletions and/or insertions of one or more nucleotides or mutations that naturally occur in bacterial and plant cell transformation.

### Plants Comprising DNA Molecules

Constructs, expression cassettes, and vectors comprising DNA molecules as disclosed herein can be constructed and introduced into a plant cell in accordance with transformation methods and techniques known in the art. For example, Agrobacterium-mediated transformation is described in U.S. Patent Application Publications 2009/0138985A1 (soybean), 2008/0280361A1 (soybean), 2009/0142837A1 (corn), 2008/0282432 (cotton), 2008/0256667 (cotton), 2003/0110531 (wheat), 2001/0042257 A1 (sugar beet), U.S. Pat. No. 5,750,871 (canola), U.S. Pat. No. 7,026,528 (wheat), and U.S. Pat. No. 6,365,807 (rice), and in Arencibia et al. (1998) Transgenic Res. 7:213-222 (sugarcane) all of which are incorporated herein by reference in their entirety. Transformed cells can be regenerated into transformed plants that express the polypeptides disclosed herein and demonstrate activity through bioassays as described herein as well as those known in the art. Plants can be derived from the plant cells by regeneration, seed, pollen, or meristem transformation techniques. Methods for transforming plants are known in the art.

The term “plant cell” or “plant” can include but is not limited to a dicotyledonous or monocotyledonous plant. In certain embodiments, plants provided herein are legumes, including, but not limited to, beans, soybeans, peas, chickpeas, peanuts, lentils, lupins, mesquite, carob, tamarind, alfalfa, and clover. Plants provided herein may also be non-legume plants, such as Parasponia, alder trees, or elm trees.

The term “plant cell” or “plant” can also include but is not limited to an alfalfa, almond, Bambara groundnut, banana, barley, bean, black currant, broccoli, cabbage, blackberry, brassica, canola, carrot, cassava, castor, cauliflower, celery, chickpea, Chinese cabbage, citrus, coconut, coffee, corn, cowpea, clover, cotton, a cucurbit, cucumber, Douglas fir, eggplant, eucalyptus, flax, garlic, forage legumes, grape, hemp, hops, indigo, leek, legume trees, lentil, lettuce, Loblolly pine, lotus, lupin, millets, melons, Medicago spp., nut, oat, olive, onion, ornamental, palm, pasture grass, pea, peach, peanut, pepper, pigeon pea, pine, potato, poplar, pumpkin, pulses, Radiata pine, radish, rapeseed, raspberry, rice, rootstocks, rye, red currant, safflower, shrub, sorghum, Southern pine, soybean, spinach, squash, strawberry, sugar beet, sugarcane, sunflower, corn, sweet gum, sweet potato, switchgrass, tea, tobacco, tomato, triticale, turf grass, walnut, watermelon, wheat, and yam. plant cell or plant.

Other plant or plant cells can be selected from plant species of interest including, but are not limited to, Arabidopsis thaliana, peppers (Capsicum spp; e.g., Capsicum annuum, C. baccatum, C. chinense, C. frutescens, C. pubescens, and the like), cowpea (Vigna unguiculata), tomatoes (Lycopersicon esculentum), tobacco (Nicotiana tabacum), eggplant (Solanum melongena), petunia (Petunia spp., e.g., Petunia×hybrida or Petunia hybrida), corn or maize (Zea mays), Brassica ssp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), green millet (Setaria viridis), finger millet (Eleusine coracana)), sunflower (Helianthus annuus), safflower (Carthamus tinctorius), wheat (Triticum aestivum), soybean (Glycine max), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), switchgrass (Panicum virgatum), algae (e.g., Chlamydomonas reinhardtii, Botryococcus braunii, Chlorella spp., Dunaliella tertiolecta, Gracilaria spp.), oats, barley, vegetables, ornamentals, and conifers. The nucleic acid molecules and polynucleotide constructs of the present invention can also be used for transformation of any algae species.

As used herein, the term “plant” refers to any living organism belonging to the kingdom Plantae (i.e., any genus/species in the Plant Kingdom). In some embodiments, the plant is a tree, herb, bush, grass, vine, fern, moss or green algae. The plant may be monocotyledonous (monocot), or dicotyledonous (dicot). Examples of particular plants that may comprise a polynucleotide of the invention include but are not limited to Arabidopsis, Brachypodium, switchgrass, corn, potato, rose, apple tree, sunflower, wheat, rice, bananas, plantains, tomatoes, opo, pumpkins, squash, lettuce, cabbage, oak trees, guzmania, geraniums, hibiscus, clematis, poinsettias, sugarcane, taro, duck weed, pine trees, Kentucky blue grass, zoysia, coconut trees, cauliflower, cavalo, collards, cowpea, kale, kohlrabi, mustard greens, rape greens, and other brassica leafy vegetable crops, bulb vegetables (e.g., garlic, leek, onion (dry bulb, green, and Welch), shallot), citrus fruits (e.g., grapefruit, lemon, lime, orange, tangerine, citrus hybrids, pummelo), cucurbit vegetables (e.g., cucumber, citron melon, edible gourds, gherkin, muskmelons (including hybrids and/or cultivars of Cucumis melons), water-melon, cantaloupe, and other cucurbit vegetable crops), fruiting vegetables (including eggplant, ground cherry, pepino, pepper, tomato, tomatillo), grape, leafy vegetables (e.g., romaine), root/tuber and corm vegetables (e.g., potato, yam, cassava, taro), tree nuts (almond, pecan, pistachio, and walnut), berries (e.g., tomatoes, barberries, currants, elderberries, gooseberries, honeysuckles, mayapples, nannyberries, Oregon-grapes, see-buckthorns, hackberries, bearberries, lingonberries, strawberries, sea grapes, blackberries, cloudberries, loganberries, raspberries, salmonberries, thimbleberries, and wineberries), cereal crops (e.g., corn (maize), rice, wheat, barley, sorghum, millets, oats, ryes, triticales, buckwheats, fonio, quinoa, oil palm), Brassicaceae family plants, and Fabaceae family plants, pome fruit (e.g., apples, pears), stone fruits (e.g., coffees, jujubes, mangos, olives, coconuts, oil palms, pistachios, almonds, apricots, cherries, damsons, nectarines, peaches and plums), vine (e.g., table grapes, wine grapes), fiber crops (e.g., hemp, cotton), ornamentals, and the like.

In certain embodiments, transgenic plants and transgenic plant parts regenerated from a transgenic plant cell are provided. In certain embodiments, the transgenic plants can be obtained from a transgenic seed, by cutting, snapping, grinding, or otherwise disassociating the part from the plant. In certain embodiments, the plant part can be a seed, a boll, a leaf, a flower, a stem, a root, or any portion thereof, or a non-regenerable portion of a transgenic plant part. As used in this context, a “non-regenerable” portion of a transgenic plant part is a portion that cannot be induced to form a whole plant or that cannot be induced to form a whole plant that is capable of sexual and/or asexual reproduction. In certain embodiments, a non-regenerable portion of a plant part is a portion of a transgenic seed, boll, leaf, flower, stem, or root.

The term “transformation” refers to the introduction of a DNA molecule into a recipient host. As used herein, the term “host” refers to bacteria, fungi, or plants, including any cells, tissues, organs, or progeny of the bacteria, fungi, or plants. Plant tissues and cells of particular interest include protoplasts, calli, roots, tubers, seeds, stems, leaves, seedlings, embryos, and pollen.

As used herein, the term “transformed” refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is inherited by subsequent progeny. A “transgenic” or “transformed” cell or organism may also include progeny of the cell or organism and progeny produced from a breeding program employing such a transgenic organism as a parent in a cross and exhibiting an altered phenotype resulting from the presence of a foreign DNA molecule. The introduced DNA molecule may also be transiently introduced into the recipient cell such that the introduced DNA molecule is not inherited by subsequent progeny. The term “transgenic” refers to a bacterium, fungus, or plant containing one or more heterologous DNA molecules.

There are many methods well known to those of skill in the art for introducing DNA molecules into plant cells. The process generally comprises the steps of selecting a suitable host cell, transforming the host cell with a vector, and obtaining the transformed host cell. Methods and materials for transforming plant cells by introducing a plant construct into a plant genome in the practice of this invention can include any of the well-known and demonstrated methods. Suitable methods include, but are not limited to, bacterial infection (e.g., Agrobacterium), binary BAC vectors, direct delivery of DNA (e.g., by PEG-mediated transformation, desiccation/inhibition-mediated DNA uptake, electroporation, agitation with silicon carbide fibers, and acceleration of DNA coated particles), gene editing (e.g., CRISPR-Cas systems), among others.

Host cells may be any cell or organism, such as a plant cell, algal cell, algae, fungal cell, fungi, bacterial cell, or insect cell. In specific embodiments, the host cells and transformed cells may include cells from crop plants.

In various embodiments, the methods described herein can involve introducing a polynucleotide construct into a plant. By “introducing” is intended presenting to the plant the polynucleotide construct in such a manner that the construct gains access to the interior of a cell of the plant. The methods of the invention do not depend on a particular method for introducing a polynucleotide construct to a plant, only that the polynucleotide construct gains access to the interior of at least one cell of the plant. Methods for introducing polynucleotide constructs into plants are known in the art including, but not limited to, stable transformation methods, transient transformation methods, and virus-mediated methods.

“Stable transformation” is intended that the polynucleotide construct introduced into a plant integrates into the genome of the plant and is capable of being inherited by progeny thereof. “Transient transformation” is intended that a polynucleotide construct introduced into a plant does not integrate into the genome of the plant.

A transgenic plant subsequently may be regenerated from a transgenic plant cell of the invention. Using conventional breeding techniques or self-pollination, seed may be produced from this transgenic plant. Such seed, and the resulting progeny plant grown from such seed, will contain the recombinant DNA molecule of the invention, and therefore will be transgenic.

Transgenic plants of the invention can be self-pollinated to provide seed for homozygous transgenic plants of the invention (homozygous for the recombinant DNA molecule) or crossed with non-transgenic plants or different transgenic plants to provide seed for heterozygous transgenic plants of the invention (heterozygous for the recombinant DNA molecule). Both such homozygous and heterozygous transgenic plants are referred to herein as “progeny plants.” Progeny plants are transgenic plants descended from the original transgenic plant and containing the recombinant DNA molecule of the invention. Seeds produced using a transgenic plant of the invention can be harvested and used to grow generations of transgenic plants, i.e., progeny plants of the invention, comprising the construct of this invention and expressing a gene of agronomic interest. Descriptions of breeding methods that are commonly used for different crops can be found in one of several reference books, see, e.g., Allard, Principles of Plant Breeding, John Wiley & Sons, NY, U. of CA, Davis, CA, 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

The transformed plants may be analyzed for the presence of the gene or genes of interest and the expression level and/or profile conferred by the regulatory elements of the invention. Those of skill in the art are aware of the numerous methods available for the analysis of transformed plants. For example, methods for plant analysis include, but are not limited to, Southern blots or northern blots, PCR-based approaches, biochemical analyses, phenotypic screening methods, field evaluations, and immunodiagnostic assays. The expression of a transcribable DNA molecule can be measured using TaqMan® (Applied Biosystems, Foster City, CA) reagents and methods as described by the manufacturer and PCR cycle times determined using the TaqMan® Testing Matrix. Alternatively, other methods and reagents for measuring expression of a transcribable DNA molecule are well known in the art. For example, the Invader® (Third Wave Technologies, Madison, WI) or SYBR Green (Thermo Fisher, A46012) reagents and methods as described by the manufacturer can be used to evaluate transgene expression.

The seeds of the plants of this invention can be harvested from fertile transgenic plants and be used to grow progeny generations of transformed plants of this invention including hybrid plant lines comprising the construct of this invention and expressing a gene of agronomic interest.

The present invention also provides for parts of the plants of the present invention. Plant parts, without limitation, include leaves, stems, roots, tubers, seeds, endosperm, ovule, and pollen. The invention also includes and provides transformed plant cells which comprise a nucleic acid molecule of the present invention.

The transgenic plant may pass along the transgenic polynucleotide molecule to its progeny. Progeny includes any regenerable plant part or seed comprising the transgene derived from an ancestor plant. The transgenic plant is preferably homozygous for the transformed polynucleotide molecule and transmits that sequence to all offspring as a result of sexual reproduction. Progeny may be grown from seeds produced by the transgenic plant. These additional plants may then be self-pollinated to generate a true breeding line of plants. Progeny from these plants are evaluated, among other things, for gene expression. The gene expression may be detected by several common methods such as western blotting, northern blotting, immuno-precipitation, and ELISA.

As an alternative to traditional transformation methods, a DNA molecule, such as a transgene, expression cassette(s), etc., may be inserted or integrated into a specific site or locus within the genome of a plant or plant cell via site-directed integration. Recombinant DNA construct(s) and molecule(s) of this disclosure may thus include a donor template sequence comprising at least one transgene, expression cassette, or other DNA sequence for insertion into the genome of the plant or plant cell. Such donor template for site-directed integration may further include one or two homology arms flanking an insertion sequence (i.e., the sequence, transgene, cassette, etc., to be inserted into the plant genome). The recombinant DNA construct(s) of this disclosure may further comprise an expression cassette(s) encoding a site-specific nuclease and/or any associated protein(s) to carry out site-directed integration. These nuclease-expressing cassette(s) may be present in the same molecule or vector as the donor template (in cis) or on a separate molecule or vector (in trans). Several methods for site-directed integration are known in the art involving different proteins (or complexes of proteins and/or guide RNA) that cut the genomic DNA to produce a double strand break (DSB) or nick at a desired genomic site or locus. Briefly as understood in the art, during the process of repairing the DSB or nick introduced by the nuclease enzyme, the donor template DNA may become integrated into the genome at the site of the DSB or nick. The presence of the homology arm(s) in the donor template may promote the adoption and targeting of the insertion sequence into the plant genome during the repair process through homologous recombination, although an insertion event may occur through non-homologous end joining (NHEJ). Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome.

### Commodity Products

The present invention provides a commodity product comprising DNA molecules according to the invention. As used herein, a “commodity product” refers to any composition or product which is comprised of material derived from a plant, seed, plant cell or plant part comprising a DNA molecule of the invention. Commodity products may be sold to consumers and may be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, flour, flakes, bran, fiber, milk, cheese, paper, cream, wine, and any other food for human consumption; and biomasses and fuel products. Viable commodity products include but are not limited to seeds and plant cells. Plants comprising a DNA molecule according to the invention can thus be used to manufacture any commodity product typically acquired from plants or parts thereof.

Having now generally described the invention, the same will be more readily understood through reference to the following examples which are provided by way of illustration and are not intended to be limiting of the present invention, unless specified. It should be appreciated by those of skill in the art that the techniques disclosed in the following examples represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention, therefore all matter set forth or shown in the accompanying drawings is to be interpreted as illustrative and not in a limiting sense.

### EXAMPLES

### Example 1

### Mt12a is Required for Nitrogen Fixation

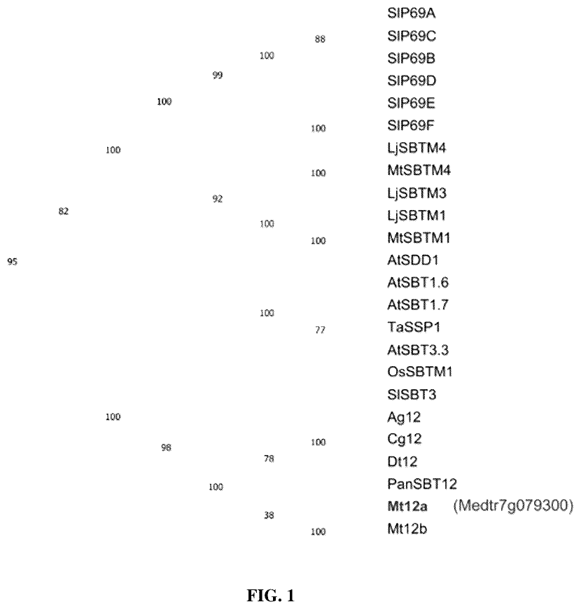

Subtilases are a large family of proteases present in hundreds of green plant species as well as numerous eukaryotes, archaea, and bacteria. A phylogenic tree of representative subtilases in plants is provide in FIG. 1.

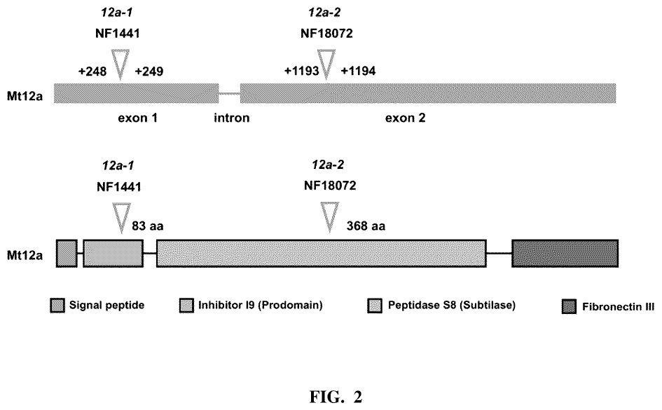

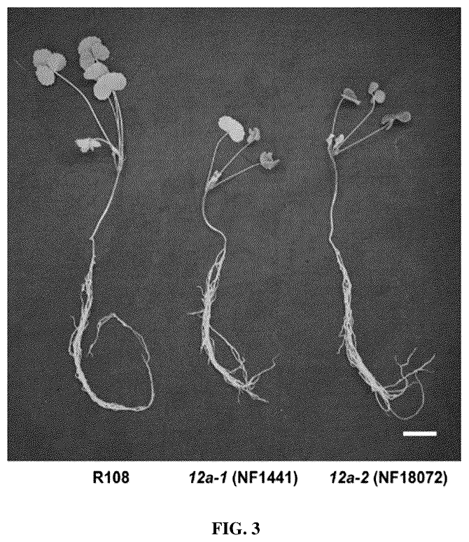

The subtilase gene Mt12a was identified in Medicago truncatula. In order to genetically assess the function of the Mt12a protein encoded by the Mt12a gene, two independent Tnt1 transposon insertion mutants were isolated from the Medicago truncatula Mutant Database (NF1441 [Mt12a-1] and NF18072 [Mt12a-2] containing an insertion at 248 bp and 1193 bp downstream of the start codon, respectively) (FIG. 2). Homozygous offspring were isolated from both lines and confirmed by genetic analysis of the insertion locus. Both homozygous Mt12a mutant plants exhibited a strong nitrogen deficiency phenotype under symbiotic conditions (FIG. 3).

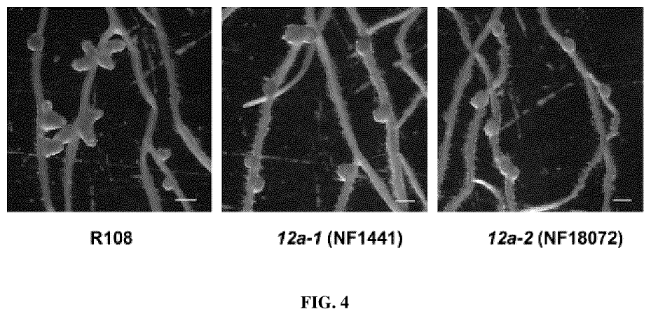

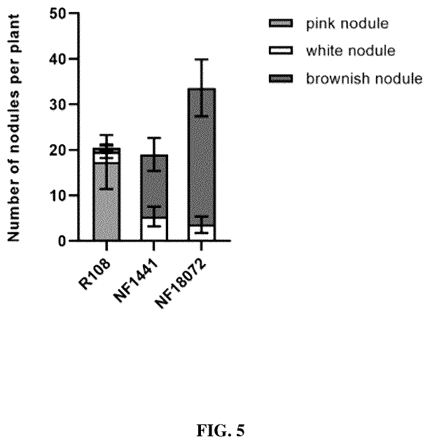

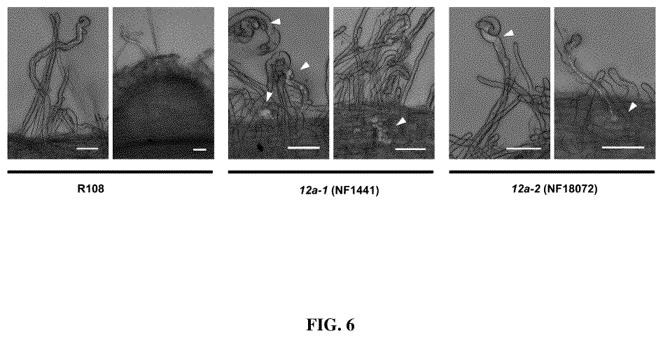

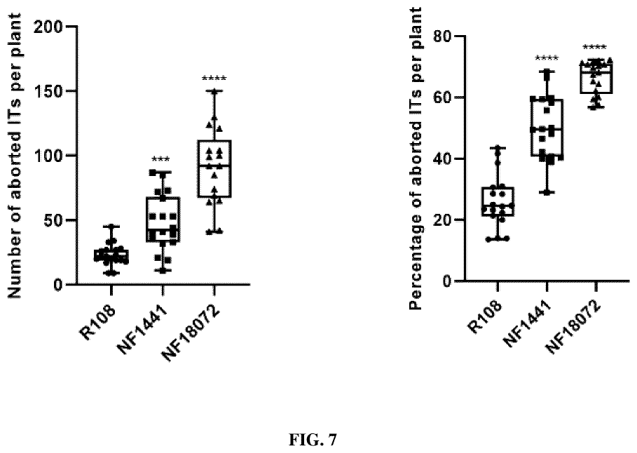

At 18 days post inoculation (dpi) with Sinorhizobium meliloti, pink and elongated nodules were observed in R108 wild type (WT) roots. By contrast, mutant plants formed only brownish or white nodules indicating that they were not functional (FIGS. 4 and 5). Mt12a was also observed to be expressed at an early stage after inoculation, and it was therefore tested whether loss function of Mt12a could influence rhizobial infection. It was determined that most of the infection threads (ITs) forming on mutant roots were aborted indicating Mt12a may also regulate early infection (FIGS. 6 and 7). Taken together, these results indicate that Mt12a is essential for nitrogen fixation.

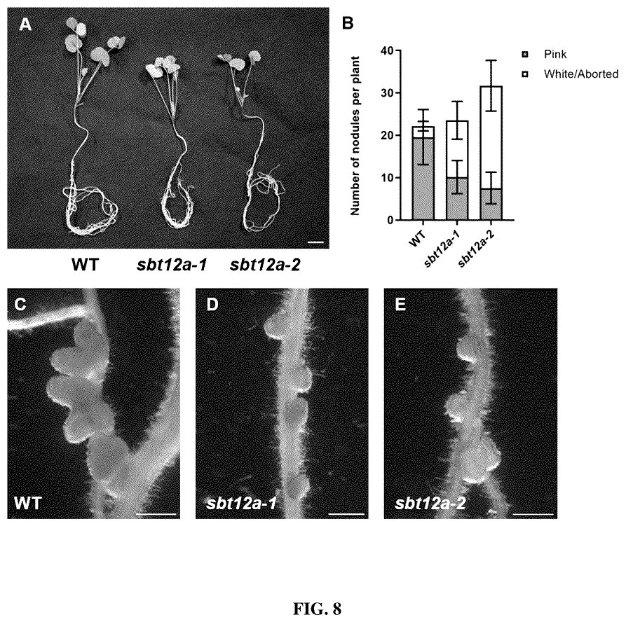

Upon seed propagation (T3), the impact of the Mt12a mutations in both alleles was analyzed. For this, plants were grown for 7 days in open pots under nitrogen deprived conditions before being inoculated with Sinorhizobium meliloti (Sm2011) for 21 days. At this time, mutant plants had developed severe symptoms of nutrient deficiency as indicated by strong anthocyanin accumulation in leaves and reduced shoot growth (FIG. 8, Panel A). Plants were then harvested and quantitatively analyzed for the presence of mature pink and immature white nodules. Both mutant alleles formed significantly less pink, elongated nodules compared to the wild-type (FIG. 8, Panels B-E).

### Example 2

### Mt12a is Required for Normal Nodule Zonation

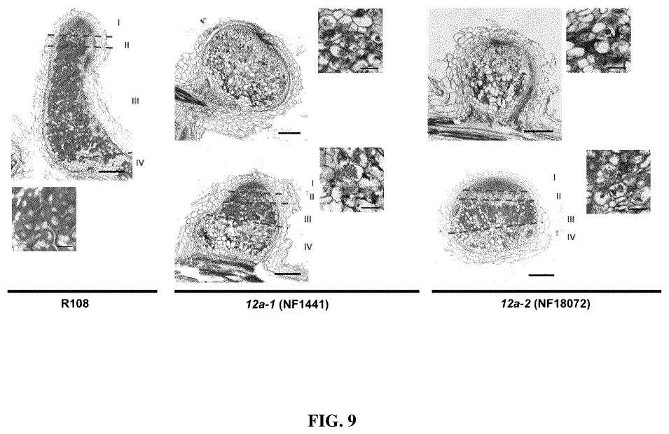

To further investigate the biological role of the Mt12a gene during symbiosis, semi-thin longitudinal nodule sections were prepared and stained with toluidine blue. In comparison with WT nodules, two types of nodules were produced from Mt12a mutants. They either contained no nitrogen-fixing cells indicating Mt12a controls rhizobial colonization or displayed modified nodule structure. In that case, the fixation zone (zone III) was drastically reduced whereas the senescence zone (zone IV) was expanded (FIG. 9). These experiments confirmed that mutant nodules developed far fewer infected cells and a largely increased senescence zone compared to the wild-type.

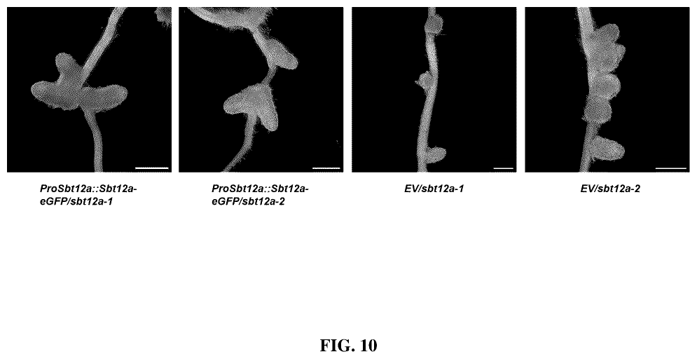

Although two independent mutant alleles showing the same phenotype were isolated, a genetic complementation experiment was conducted to confirm these results. For this, Mt12a-1 and Mt12a-2 mutant roots were transformed with a construct, driving the expression of a Mt12a-GFP fusion from the Mt12a promoter (ProSbt12a::Sbt12a-GFP). Indeed, wild-type-like nodule patterns were restored in complemented Mt12a mutant roots (FIG. 10), demonstrating that the insertions in the Mt12a gene in both alleles were causative for the observed nodulation phenotype.

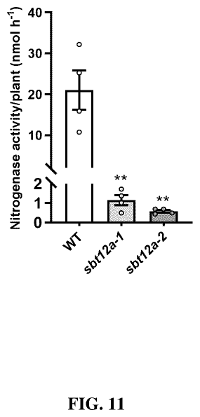

To test whether this morphological phenotype correlates with decreased nitrogen fixation capacity of the plants as indicated by the above-ground phenotypes, an acetylene reduction assay was conducted that enabled quantification of rates of nitrogen fixation. Indeed, nitrogenase activity was significantly reduced in both mutant alleles (FIG. 11).

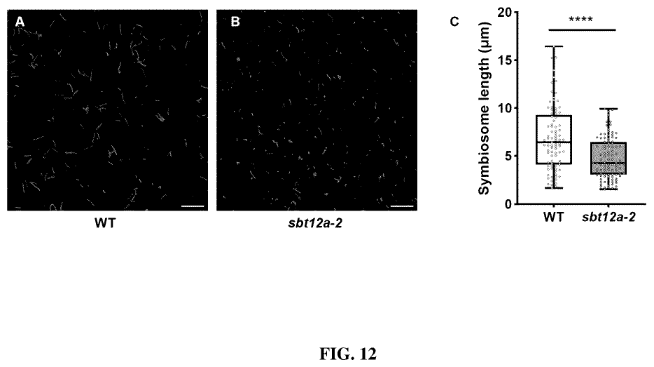

To finally assess whether these mutants also show alterations in symbiosome morphology, intact symbiosome was isolated by differential centrifugation and scored for length in the Mt12a-2 mutant. It was found that symbiosome sizes were significantly lower in this genotype (FIG. 12, Panels A-C).

### Example 3

### Mt12a Shows an Infection-Specific Expression Pattern

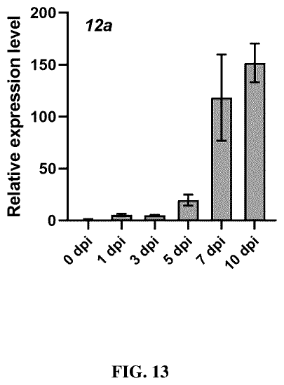

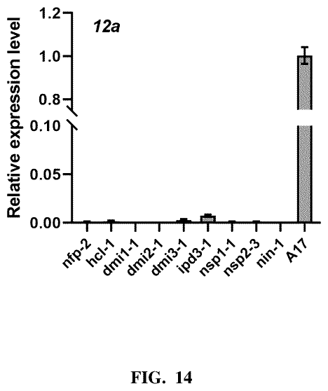

Subtilase Mt12a (Medtr7g079300; SEQ ID NO: 3) was evaluated for expression levels following rhizobial inoculation. In order to determine this, qRT-PCR was performed to check the expression of Mt12a at different time points following rhizobial treatment. The results revealed that Mt12a was significantly up-regulated and continued to increase over the timepoints tested (FIG. 13). However, this induced transcriptional pattern was strongly impaired in different M. truncatula mutants, including nin (FIG. 14). These results indicate that Mt12a may act downstream of a symbiotic signaling pathway.

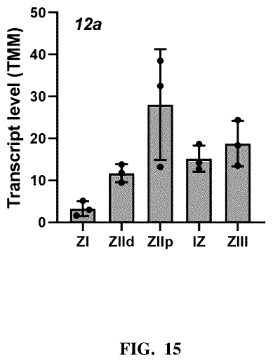

In nodules, Mt12a transcript was highly accumulated in zone II and zone III (Carrere et al., 2021) (FIG. 15). To determine the spatial and temporal expression pattern, the 2 kb Mt12a promoter element (ProMt12a; SEQ ID NO:4) was fused to a 0-glucuronidase (GUS) sequence and transformed into roots.

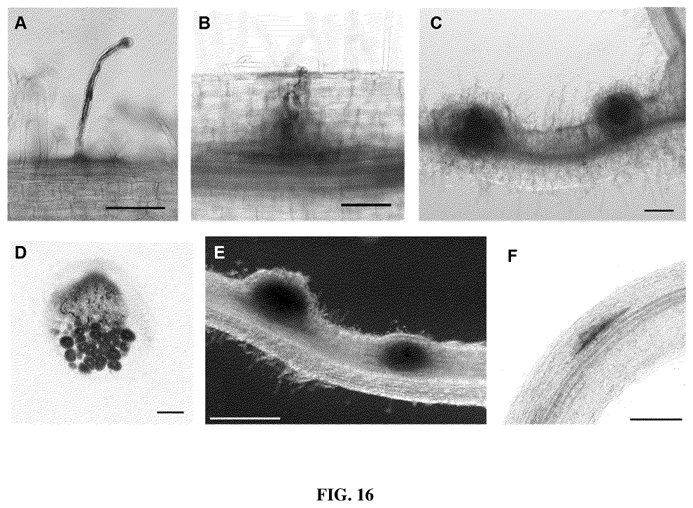

When transgenic M. truncatula hairy roots carrying this construct were inoculated with symbiotic S. meliloti for 7 and 14 days and subsequently stained for GUS-activity, it was confirmed that the Mt12a-promoter was specifically induced in infected trichoblast cells, along the infection thread trajectory, in nodule primordia and within the infection zone of indeterminate M. truncatula nodules (FIG. 16, Panels A-D).

Using cg12 protein sequences from Casuarina glauca as an input, a second Medicago gene, Mt12b (Medtr7g079310), was identified that is closely related to Mt12a. Using publicly available transcript data, it was found that Mt12b expression is not only induced in nodules but also correlates with lateral root formation. This was confirmed by creating a reporter, where the GUS-gene was expressed under the control of a 2000 bp long fragment of the putative Mt12b-promoter (ProSbt12b:GUS) (FIG. 16, Panels E, F). In the past, few other subtilases have been described to be transcriptionally regulated under symbiotic conditions. In Lotus japonicus, SbtM1, SbtM3, and SbtM4 were shown to be induced during AMS in a common symbiosis pathway (CSP)-dependent manner, while SbtM4 and another subtilase named SbtS were also highly upregulated during RNS. Knock down of SbtM1 and SbtM3 lead to impaired AM development, but appear not to affect nodulation process (Takeda et al., 2009, Takeda et al., 2011). However, despite these elaborative studies, no genetic evidence demonstrating the involvement of subtilases in RNS has been reported to date.

### Example 4

### Mt12a Localizes into Symbiotic Infected Cells

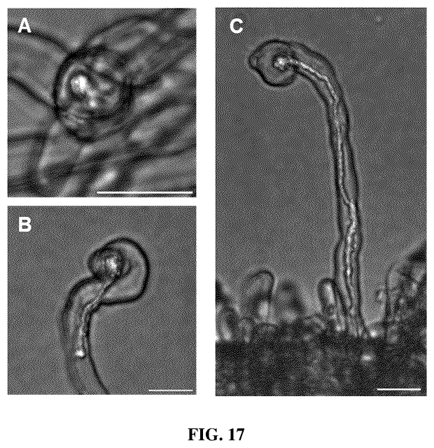

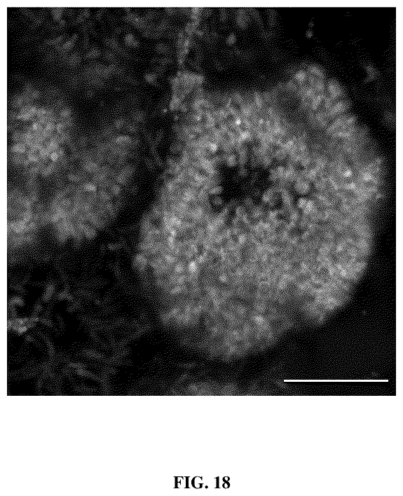

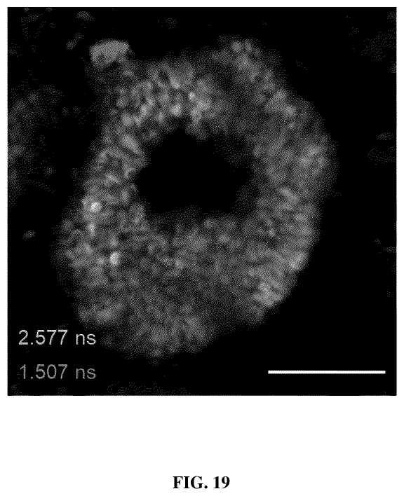

To further investigate the spatial and temporal expression profile of the Mt12a promoter, a translational Mt12a-GFP fusion construct was generated and transformed into plant roots which were then inoculated with mCherry-labeled S. meliloti. At early stages, robust Mt12a-GFP signal was observed in root hair cells containing rhizobia (FIG. 17, Panels A-C), while in nodules the fluorescence was concentrated in fixing cells and closely associated with rhizobia (FIG. 18). To unambiguously distinguish the real signal from autofluorescence, we applied Fluorescent Lifetime Imaging Microscopy (FLIM) to further confirm the localization of Mt12a protein (FIG. 19).

### Example 5

### Identification of Substrates of Mt12a

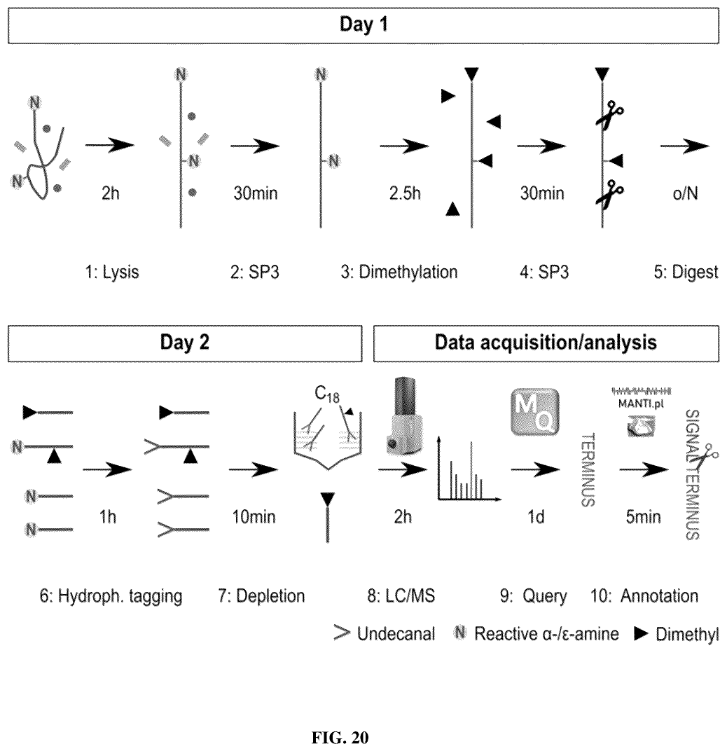

In order to identify direct protein targets of Mt12a, a High-efficiency Undecanal-based N-Termini Enrichment (HUNTER) approach was used (Demir et al., 2022). This method allows the N-termini of proteins to be enriched, which provides unique information on proteolytically processed or N-terminally modified proteoforms (FIG. 20). The candidate substrates identified from HUNTER were then further tested.

To better prepare for putative candidates deriving from this analysis and to identify genetic pathways that are dependent on the presence of Mt12a, bulk RNA sequencing was performed on Mt12a mutant and wildtype nodules with S. meliloti. In this experiment, 1611 and 1142 genes were identified as being up- or downregulated in the Mt12a mutants, respectively. Using GO-term analysis, it was determined that multiple members of the NCR family (also annotated as metal ion binding; GO:0046872) were among the genes being most significantly downregulated in the mutants. These results match well phenotypical data showing alterations in symbiosome formation and an enlarged senescence zone in Mt12a mutant nodules. These data further emphasize the strong impact of Mt12a on symbiosome differentiation and integrity. Taken together, these data unambiguously demonstrate that Mt12a is required for successful nodule function and thus provide the first genetic evidence for the importance of a single subtilase in root symbiotic interactions.

### Example 6

### Transcriptional Activation of Mt12a Gene by NODULE INCEPTION (NIN)

The qRT-PCR results described above indicate that Mt12a expression was hardly detected in nin-1 mutant background (FIGS. 10 and 11), suggesting that Mt12a may be directly regulated by NIN. To test this, chromatin immunoprecipitation (ChIP), electrophoresis mobility shift assay (EMSA) as well as transactivation assay are performed.

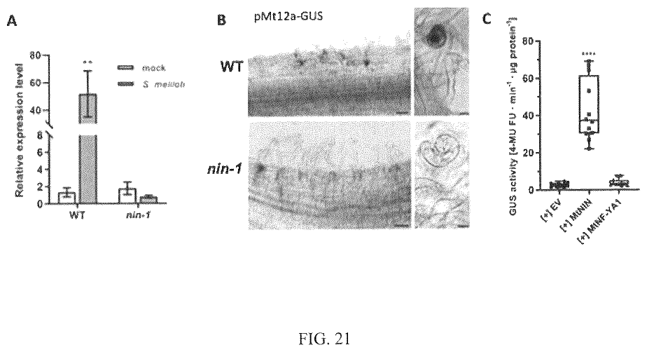

RealTime PCR experiments were performed to determine whether expression of MT12a is NIN-dependent. FIG. 21, Panel A, shows that Mt12a expression is not induced in the nin-1 mutant upon inoculation with S. meliloti. As shown in FIG. 21, Panel B, the promoter of Mt12a is not activated above background levels in the nin-1 mutant. Transactivation assay conducted in N. benthamiana leaves shows activation of the ProSbt12a:GUS reporter upon co-expression with NIN but not with NF-YA1.

### Example 7

### Overexpression of Mt12a in Transgenic Plants

A recombinant construct comprising a nucleotide sequence encoding the Mt12a protein is transformed into protoplasts or plant cells, which are regenerated to produce transgenic plants overexpressing Mt12a protein. Transgenic protoplasts and plants are inoculated with rhizobia and evaluated phenotypically as described herein including morphological changes related to enhanced symbiotic infection such as increased nodule number per plant and increased infection threads per plant. Plants overexpressing Mt12a exhibit altered symbiotic infection compared with plants not comprising the recombinant Mt12a constructs.

### Example 8

### Overexpression of Protein in Transgenic Plants Using the Mt12a Promoter

A recombinant construct comprising the Mt12a promoter operably linked to a heterologous transcribable polynucleotide is transformed into protoplasts or plant cells, which are regenerated to produce transgenic plants overexpressing the heterologous protein. Transgenic protoplasts and plants are evaluated phenotypically as described herein including morphological changes related to enhanced symbiotic infection such as increased nodule number per plant and increased infection threads per plant.

Having illustrated and described the principles of the invention, it should be apparent to persons skilled in the art that the invention can be modified in arrangement and detail without departing from such principles. We claim all modifications that are within the spirit and scope of the claims. All publications and published patent documents cited herein are hereby incorporated by reference to the same extent as if each individual publication or patent application is specifically and individually indicated to be incorporated by reference.

What is claimed is:

1. A recombinant DNA molecule comprising a heterologous promoter operably linked to a polynucleotide segment encoding a subtilase protein, homolog, or fragment thereof, wherein:
a) said protein comprises an amino acid sequence of SEQ ID NO:3;
b) said protein comprises an amino acid sequence having at least 70%, at least 75%, at least 80%, at least 85%, at least 90%, at least 95%, at least 98%, at least 99%, or about 100% sequence identity to SEQ ID NO:3; or
c) said polynucleotide segment hybridizes under stringent hybridization conditions to a polynucleotide having the nucleotide sequence of SEQ ID NO: 3.

2. The recombinant DNA molecule of claim 1, wherein:
a) said recombinant DNA molecule is expressed in a plant cell to produce an increase in symbiotic nitrogen fixation; or
b) said recombinant DNA molecule is in operable linkage with a vector, and said vector is selected from the group consisting of a plasmid, phagemid, bacmid, cosmid, and a bacterial or yeast artificial chromosome.

3. The recombinant DNA molecule of claim 1, wherein the subtilase protein having at least 70% sequence identity to SEQ ID NO:3 has subtilase activity.

4. The recombinant DNA molecule of claim 1, present within a host cell, wherein said host cell is selected from the group consisting of a bacterial host cell and a plant cell.

5. The recombinant DNA molecule of claim 4, wherein said bacterial host cell is from a genus of bacteria selected from the group consisting of: Agrobacterium, Rhizobium, Bacillus, Brevibacillus, Escherichia, Pseudomonas, Klebsiella, Pantoea, and Erwinia.

6. The recombinant DNA molecule of claim 5, wherein said Bacillus is Bacillus cereus or Bacillus thuringiensis, said Brevibacillus is a Brevibacillus laterosperous, and said Escherichia is Escherichia coli.

7. The recombinant DNA molecule of claim 4, wherein said plant cell is a dicotyledonous or a monocotyledonous plant cell.

8. The recombinant DNA molecule of claim 7, wherein said plant cell is selected from the group consisting of an alfalfa, almond, Bambara groundnut, banana, barley, bean, black currant, broccoli, cabbage, blackberry, brassica, canola, carrot, cassava, castor, cauliflower, celery, chickpea, Chinese cabbage, citrus, coconut, coffee, corn, cowpea, clover, cotton, a cucurbit, cucumber, Douglas fir, eggplant, eucalyptus, flax, garlic, forage legumes, grape, hemp, hops, indigo, leek, legume trees, lentil, lettuce, Loblolly pine, lotus, lupin, millets, melons, Medicago spp., nut, oat, olive, onion, ornamental, palm, pasture grass, pea, peach, peanut, pepper, pigeon pea, pine, potato, poplar, pumpkin, pulses, Radiata pine, radish, rapeseed, raspberry, rice, rootstocks, rye, red currant, safflower, shrub, sorghum, Southern pine, soybean, spinach, squash, strawberry, sugar beet, sugarcane, sunflower, corn, sweet gum, sweet potato, switchgrass, tea, tobacco, tomato, triticale, turf grass, walnut, watermelon, wheat, and yam plant cells.

9. A plant, a plant part, or a plant cell comprising the recombinant DNA molecule of claim 1.

10. The plant, a plant part, or a plant cell of claim 9, wherein said plant is a monocot plant or a dicot plant.

11. The plant or part thereof of claim 10, wherein said plant is selected from the group consisting of an alfalfa, almond, Bambara groundnut, banana, barley, bean, black currant, broccoli, cabbage, blackberry, brassica, canola, carrot, cassava, castor, cauliflower, celery, chickpea, Chinese cabbage, citrus, coconut, coffee, corn, cowpea, clover, cotton, a cucurbit, cucumber, Douglas fir, eggplant, eucalyptus, flax, garlic, forage legumes, grape, hemp, hops, indigo, leek, legume trees, lentil, lettuce, Loblolly pine, lotus, lupin, millets, melons, Medicago spp., nut, oat, olive, onion, ornamental, palm, pasture grass, pea, peach, peanut, pepper, pigeon pea, pine, potato, poplar, pumpkin, pulses, Radiata pine, radish, rapeseed, raspberry, rice, rootstocks, rye, red currant, safflower, shrub, sorghum, Southern pine, soybean, spinach, squash, strawberry, sugar beet, sugarcane, sunflower, corn, sweet gum, sweet potato, switchgrass, tea, tobacco, tomato, triticale, turf grass, walnut, watermelon, wheat, and yam.

12. A transgenic seed comprising the recombinant DNA molecule of claim 1.

13. A method of producing progeny seed comprising the recombinant DNA molecule of claim 1, the method comprising:
a) planting a first seed comprising the recombinant DNA molecule of claim 1;
b) growing a plant from the seed of step a; and
c) harvesting the progeny seed from the plants, wherein said harvested seed comprises said recombinant DNA molecule.

14. A plant with improved symbiotic nitrogen fixation, wherein the cells of said plant comprise the recombinant DNA molecule of claim 1.

15. A method for increasing symbiotic nitrogen fixation in a plant, said method comprising:
a) expressing a subtilase protein or fragment or variant thereof as set forth in SEQ ID NO:3 or at least 70% identical to SEQ ID NO: 3 having subtilase activity in a plant; and
b) contacting said plant with an effective amount of one or more rhizobia bacterium, arbuscular mycorrhiza fungi, or a combination thereof.

16. The method of claim 15, wherein:
a) said rhizobia bacterium is selected from the group consisting of: S. meliloti, Mesorhizobium loti, Sinorhizobium meliloti, Sinorhizobium fredii, Rhizobium sp. IRBG74 and NGR234, Bradyrhizobium sp.; or
b) said arbuscular mycorrhiza fungi is selected from the group consisting of: R. irregularis, Rhizophagus intraradices, Glomus mosseae, and Funneliformis mosseae.

17. A recombinant DNA molecule comprising a polynucleotide sequence selected from the group consisting of:
a) a sequence with at least 70 percent sequence identity to SEQ ID NO:4;
b) a sequence comprising SEQ ID NO:4;
c) a fragment of a sequence having at least 85 percent sequence identity to SEQ ID NO:4, wherein the fragment has gene-regulatory activity; and
d) a fragment of SEQ ID NO:4, wherein the fragment has gene-regulatory activity;
wherein said sequence is operably linked to a heterologous transcribable polynucleotide molecule.

18. The DNA molecule of claim 17, wherein the DNA molecule is active as a promoter.

19. The DNA molecule of claim 17, wherein the DNA molecule comprises a heterologous regulatory element.

20. The recombinant DNA molecule of claim 17, wherein the sequence provides expression of said heterologous transcribable polynucleotide molecule in response to an external stimulus.

21. The recombinant DNA molecule of claim 20, wherein said external stimulus is a presence of a rhizobia bacterium.

22. A transgenic plant cell comprising the recombinant DNA molecule of claim 17.

23. A transgenic plant, plant part, or seed, comprising the recombinant DNA molecule of claim 17.

24. A progeny plant of the transgenic plant of claim 23, or a part thereof, wherein the progeny plant or part thereof comprises said recombinant DNA molecule.

25. A method of expressing a transcribable polynucleotide molecule comprising obtaining a transgenic plant according to claim 23 and cultivating the plant, wherein the transcribable polynucleotide is expressed.

26. A construct comprising at least one copy of a DNA molecule of claim 17, and an operably linked transcribable gene of agronomic interest.

27. The construct of claim 26, wherein the construct comprises, in the 5′-3′ direction: (a) the at least one copy of said DNA molecule; (b) the operably linked transcribable gene of agronomic interest; and (c) a gene termination sequence.

28. The construct of claim 26, wherein the transcribable gene of agronomic interest comprises an open reading frame encoding a polypeptide.

29. A method of expressing a gene of agronomic interest in a plant or plant cell, the method comprising incorporating into a plant cell, a construct comprising the DNA molecule of claim 17, operably linked to a transcribable gene of agronomic interest, wherein the DNA molecule is capable of driving the expression of the operably linked gene of agronomic interest in the plant cell.

30. The method of claim 29, further comprising regenerating a transformed plant from said plant cell.

31. The method of claim 29, wherein said plant cell is stably transformed with said construct.

32. A method of producing a transgenic plant cell comprising introducing the DNA molecule of claim 17 into a plant cell.

33. The method of claim 32, wherein introducing said DNA molecule into said plant cell comprises transformation.

34. The method of claim 33, further comprising regenerating a transgenic plant from said plant cell.

35. The method of claim 32, wherein introducing said DNA molecule into said plant cell comprises crossing the transgenic plant of claim 23 with another plant to produce a progeny plant comprising said plant cell.
